(19) United States
(12) Patent Application Publication
(10) Pub. No.: US 2026/0000551 A1
(43) Pub. Date: Jan. 1, 2026

(54) RADIAL PNEUMATIC DISTRIBUTION SYSTEMS AND METHODS USEFUL FOR MAKING SIDE SEAMS ON COMPONENTS OF ABSORBENT ARTICLES

(71) Applicant: The Procter & Gamble Company, Cincinnati, OH (US)

(72) Inventors: Bradley William JONES, Maineville, OH (US); Michael Devin LONG, Harrison Township, OH (US); Paul Anthony VAN DER SLUIJS, Terrace Park, OH (US); Paul Anthony KAWKA, Cleves, OH (US)

(21) Appl. No.: 18/754,348

(22) Filed: Jun. 26, 2024

(51) Int. Cl. A61F 13/15

(52) U.S. Cl. CPC A61F 13/15658; A61F 2013/15861; A61F 2013/15934

(57) ABSTRACT

The present disclosure provides radial pneumatic distribution systems, methods, and apparatuses, for example, drums, configured to bond web materials together utilizing a rotating drum having fluid nozzles and fluid flow sensors associated with the housing of the drum and configured to rotate with the drum about a central longitudinal axis as the drum rotates.

### FIELD

The present disclosure relates to radial pneumatic distribution systems and methods for manufacturing absorbent articles, and more particularly, to an apparatus, for example, a drum and methods for bonding materials, such as web materials and/or components of absorbent articles and/or absorbent articles themselves, for example, to create seams, such as side seams, on the web materials and/or components of absorbent articles and/or absorbent articles using the radial pneumatic distribution system and/or apparatus.

### BACKGROUND

Along an assembly line, various types of articles, such as diapers and other absorbent articles, may be assembled by adding components to and/or otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. In some cases, individual components created from an advancing web or webs are combined with other individual components created from another advancing web or webs. Webs of material and/or component parts used to manufacture absorbent articles, for example, diapers such as pant diapers may include: backsheets, topsheets, leg cuffs, waist bands, absorbent core components, front and/or back ears, fastening components, and various types of elastic webs and components, such as leg elastics, barrier leg cuff elastics, stretch side panels, and waist elastics. Once the desired web materials and/or component parts are assembled into absorbent articles, the advancing absorbent articles are subjected to a final cut, for example, a knife cut to separate the advancing absorbent articles into discrete absorbent articles, such as diapers, for example, pant diapers, or other types of absorbent articles.

During the assembly process, various component parts and/or advancing webs of material may be bonded together, for example, seamed together in various ways. For example, advancing webs of material and/or component parts may be bonded together to ultimately create side seams on absorbent articles, for example, diapers. In some processes, advancing webs of material and/or component parts may be bonded with adhesives and/or with ultrasonic bonding apparatuses. In other processes, advancing webs and/or component parts may be mechanically bonded together with heat and pressure with or without the use of adhesives. In yet other processes, portions of advancing webs of material and/or component parts may be partially melted with a fluid, such as air, for example, hot air, and then optionally pressed together with a press member and/or corresponding anvil roll, wherein the press member and/or anvil roll may include a pattern, for example, a pattern comprising a plurality of relatively small discrete pattern elements. Thus, each side seam created with such seaming processes, for example, hot air seaming processes, includes a bond site, for example, a plurality of discrete, relatively small, bond sites, along the length of each side seam. However, some present apparatuses, such as rotating apparatuses, for example, a rotating drum and processes, such as fluid seaming processes, for example, air processes, such as hot air processes, used to create side seams with bonds may have certain disadvantages. For example, fluid nozzles used to deliver the fluid, for example, air, to the advancing web materials and/or component parts of absorbent articles and/or absorbent articles (hereafter individually and/or collectively referred to as “web”) to create the seams may exhibit fluid flow rates that are unacceptable, either too high or too low of fluid flow rates compared to an expected fluid flow rate, due to various possible reasons such as clogging or leakages in one or more of the fluid nozzles within the apparatuses. Such problems may result in too weak of seams causing failure, for example, delamination at the seam in the webs to occur during further processing and/or use by a consumer and/or too strong of seams causing unpleasant stiffness and/or roughness at the seam in the web, which may cause irritation when worn by a consumer, for example, a baby or toddler. However, currently there is no way to effectively and accurately determine which of the fluid nozzles within the drum is performing unacceptably nor where the mis-performing fluid nozzles and/or associated fluid pathways are located within the drum with respect to a central longitudinal axis about which the drum rotates during operation nor which web bonded and/or seamed on the drum is affected by the mis-performing fluid nozzles and contains a defective bond and/or seam.

Consequently, it would be beneficial to provide a radial pneumatic distribution system, a method, and an apparatus, for example, a rotating apparatus, such as a drum, for utilizing fluid seaming methods to bond an advancing web to create seams, such as side seams, in the web and ultimately in an absorbent article containing the web.

### SUMMARY

The present disclosure addresses the problems described above by providing a radial pneumatic distribution system, a method utilizing the radial pneumatic distribution system, and an apparatus, such as a drum, utilized in the radial pneumatic distribution system and method, and configured to rotate about a central longitudinal axis, to provide fluid flow rate data linked to an individual fluid nozzle or group of two or more fluid nozzles within the drum while the drum is rotating, and to bond and/or seam an advancing web, for example a web or webs that are a component of an absorbent article, such as a diaper, to create seams, for example, side seams, in the web. The radial pneumatic distribution system, method, and apparatus may be useful in utilizing a fluid passing out of the apparatus, for example, a drum (positive fluid flow) and/or passing into the apparatus, for example, a drum (negative fluid flow, such as a vacuum) to and/or through a web residing on an outer circumferential surface of the apparatus such that the web is sufficiently softened and/or partially melted to be susceptible to bonding at the softened and/or partially melted region to create a seam, such as a side seam. The physical bonding operation may at least partially occur after the web material and/or component and/or absorbent article is advanced into a nip formed by the apparatus and an anvil roll and/or off of the apparatus into a nip formed by an anvil roll and a press member separate and away from the apparatus. Alternatively, and/or in addition, the physical bonding may at least partially occur while the web material and/or component and/or absorbent article are residing on the outer surface of the apparatus, which may comprise a press member within the apparatus that activates to compress the web material and/or component and/or absorbent article between the press member of the apparatus and an anvil roll.

The apparatus, for example, a drum, includes fluid nozzles and fluid flow sensors fluidly connected to the fluid nozzles. In operation, web materials, for example, a first web material and a second web material, and/or component parts of an absorbent article and/or absorbent articles may be advanced in a machine direction onto the drum's outer circumferential surface as the drum rotates. A fluid, such as air, which may be heated to a temperature sufficient to partially melt one or more of the web materials and/or the component parts of an absorbent article and/or the absorbent articles themselves, is supplied to the rotating drum and delivered to the web materials and/or the component parts and/or the absorbent articles by the fluid nozzles, which are associated with the rotating drum. As the drum rotates, the softened and/or partially melted portion of the advancing web is compressed to form a bond, for example, a side seam. The compression occurs within a nip formed between an anvil roll and the outer circumferential surface of the drum and/or between an anvil roll and a press member within the drum that moves radially outward from the rotating drum's internal volume and retracts radially back into the internal volume of the drum after performing its compression function and/or between an anvil roll and a press member separate from and outside of the drum.

In accordance with a first aspect, a method is provided, comprising: providing a drum comprising a housing configured to rotate about a central longitudinal axis and defining an outer circumferential surface and an internal volume of the drum, fluid nozzles associated with the housing and receiving fluid from a fluid source, and fluid flow sensors associated with the housing, wherein the housing, the fluid nozzles, and the fluid flow sensors are configured to rotate about the central longitudinal axis; rotating the housing about the central longitudinal axis; transmitting a fluid from the fluid source to one or more first fluid nozzles of the fluid nozzles; monitoring a first fluid flow rate of the fluid transmitted to the one or more first fluid nozzles via a first fluid flow sensor of the fluid flow sensors as the one or more first fluid nozzles rotate about the central longitudinal axis; generating a first output corresponding to the first fluid flow rate via the first fluid flow sensor; receiving by a data processing unit the first output corresponding to the first fluid flow rate from the first fluid flow sensor; and monitoring via the data processing unit the first fluid flow rate of the fluid transmitted to the one or more first fluid nozzles using the first output. In one embodiment, the method is suitable for determining a fluid flow rate in a drum as it rotates.

The method may further comprise: transferring the first output from the first fluid flow sensor to a data transfer unit coupled to the housing, which then transfers the first output to the data processing unit.

The method may further comprise: detecting an angular position of the one or more first fluid nozzles using an angular position sensor associated with the housing as the one or more first fluid nozzles rotate about the central longitudinal axis; receiving by the data processing unit the angular position data corresponding to the angular position of the one or more first fluid nozzles as the one or more first fluid nozzles rotate about the central longitudinal axis; the monitoring comprises determining via the data processing unit the first fluid flow rate of the fluid transmitted to the one or more first fluid nozzles at each of a plurality of angular positions of the one or more first fluid nozzles that fall within a predefined angular data collection range.

The method may further comprise: comparing the first fluid flow rate of the fluid transmitted to the one or more first fluid nozzles to an expected fluid flow rate range in the data processing unit to determine when the first fluid flow rate of the fluid transmitted to the one or more first fluid nozzles falls outside of the expected fluid flow rate range.

The method may further comprise: alerting a user when the first fluid flow rate of the fluid transmitted to the one or more first fluid nozzles falls outside of the expected fluid flow rate range.

The method may further comprise: generating a flow control signal and sending the flow control signal using the data processing unit to a valve to adjust the first fluid flow rate of the fluid transmitted to the one or more first fluid nozzles when the first fluid flow rate of the fluid transmitted to the one or more first fluid nozzles falls outside of the expected fluid flow rate range.

The method may further comprise: adjusting the first fluid flow rate of the fluid transmitted to the one or more first fluid nozzles by increasing or decreasing the first fluid flow rate of the fluid transmitted to the one or more first fluid nozzles when the first fluid flow rate of the fluid transmitted to the one or more first fluid nozzles falls outside of the expected fluid flow rate range.

The method may further comprise: stopping the flow of the fluid transmitted to the one or more first fluid nozzles when the first fluid flow rate of the fluid transmitted to the one or more first fluid nozzles falls outside of the expected fluid flow rate range.

The method may further comprise: stopping the rotation of the housing about the central longitudinal axis when the first fluid flow rate of the fluid transmitted to the one or more first fluid nozzles falls outside of the expected fluid flow rate range.

The one or more first fluid nozzles may comprise a single first fluid nozzle, wherein the monitoring via the first fluid flow sensor comprises monitoring the first fluid flow rate of the fluid transmitted to the single first fluid nozzle via the first fluid flow sensor.

The one or more first fluid nozzles may comprise a pair of first fluid nozzles, wherein the monitoring via the first fluid flow sensor comprises monitoring via the first fluid flow sensor the first fluid flow rate of the fluid transmitted to the pair of first fluid nozzles prior to the fluid reaching the pair of first fluid nozzles such that a rate of fluid flow passing through each of the pair of first fluid nozzles is normally less than the first fluid flow rate sensed by the first fluid flow sensor.

The method may further comprise: transmitting the fluid to one or more second fluid nozzles of the one or more fluid nozzles; monitoring a second fluid flow rate of the fluid transmitted to the one or more second fluid nozzles via a second fluid flow sensor of the fluid flow sensors as the one or more second fluid nozzles rotate about the central longitudinal axis; generating a second output corresponding to the second fluid flow rate via the second fluid flow sensor; transferring the second output to the data processing unit; and determining via the data processing unit the second fluid flow rate of the fluid transmitted to the one or more second fluid nozzles using the second output.

The method may further comprise: detecting an angular position of the one or more second fluid nozzles using the angular position sensor associated with the housing as the one or more second fluid nozzles rotate about the central longitudinal axis; receiving by the data processing unit the angular position sensor data corresponding to the angular position of the one or more second fluid nozzles as the one or more second fluid nozzles rotate about the central longitudinal axis; the determining comprises determining via the data processing unit the second fluid flow rate of the fluid transmitted to the one or more second fluid nozzles at each of a plurality of angular positions of the one or more second fluid nozzles that fall within a predefined angular data collection range.

In accordance with a second aspect, a method is provided, comprising: providing a drum comprising a housing configured to rotate about a central longitudinal axis and defining an outer circumferential surface and an internal volume, fluid nozzles associated with the housing and receiving fluid from a fluid source, and fluid flow sensors associated with the housing, wherein the housing, the fluid nozzles, and the fluid flow sensors are configured to rotate about the central longitudinal axis; rotating the housing about the central longitudinal axis; transmitting a fluid from the fluid source to one or more first fluid nozzles of the fluid nozzles; monitoring a first fluid flow rate of the fluid transmitted to one of the first fluid nozzles via a first fluid flow sensor of the fluid flow sensors as the one of the first fluid nozzles rotates about the central longitudinal axis; monitoring a second fluid flow rate of the fluid transmitted to another one of the first fluid nozzles via a second fluid flow sensor of the fluid flow sensors as the other of the first fluid nozzles rotates about the central longitudinal axis; generating first and second outputs corresponding to the first and second fluid flow rates via the first and second fluid flow sensors; receiving by a data processing unit the first and second outputs; and monitoring, via the data processing unit, first and second fluid flow rates using the first and second outputs. In one embodiment, the method is suitable for measuring a fluid flow rate of a fluid in a drum for manufacturing absorbent articles as the drum rotates.

The one and the other first fluid nozzle may be located generally at the same angular position. In such an embodiment, the method may further comprise: detecting an angular position of the one and the other first fluid nozzle using an angular position sensor associated with the housing as the one and the other first fluid nozzle rotate about the central longitudinal axis; receiving by the data processing unit the angular position sensor data corresponding to the angular position of the one and the other first fluid nozzle as the one and the other first fluid nozzle rotate about the central longitudinal axis; the monitoring comprises determining, via the data processing unit, the first and second fluid flow rates of the fluid transmitted to the one and the other first fluid nozzle at each of a plurality of angular positions of the one and the other first fluid nozzle that fall within a predefined angular data collection range.

In accordance with a third aspect, an apparatus, for example, a drum is provided, comprising a housing that defines an outer circumferential surface and an internal volume of the drum, wherein a plurality of fluid nozzles and a plurality of fluid flow sensors each in fluid communication with at least one of the plurality of fluid nozzles are associated with the housing and wherein the outer circumferential surface comprises a plurality of apertures associated with the plurality of fluid nozzles.

The plurality of fluid nozzles may comprise two or more groups of one or more fluid nozzles supported by the housing, wherein the two or more groups of one or more fluid nozzles are spaced apart from one another about the housing and wherein each of the groups of one or more fluid nozzles is associated with one of the plurality of apertures.

The plurality of fluid nozzles may comprise a first group of two or more fluid nozzles comprising a first fluid nozzle and a second fluid nozzle, wherein one of the plurality of fluid flow sensors is associated with the first fluid nozzle and the second fluid nozzle.

The plurality of fluid nozzles may comprise a first group of two or more fluid nozzles comprising a first fluid nozzle and a second fluid nozzle, wherein the plurality of fluid flow sensors comprises a first fluid flow sensor associated with the first fluid nozzle and configured to detect a fluid flow rate of fluid flowing to the first fluid nozzle and a second fluid flow sensor associated with the second fluid nozzle and configured to detect a flow rate of fluid flowing to the second fluid nozzle.

The plurality of fluid flow sensors may comprise two or more groups of one or more fluid flow sensors, each of the two or more groups of the one or more fluid flow sensors being associated with a corresponding one of the groups of the one or more fluid nozzles.

In accordance with a fourth aspect, a method is provided, comprising: providing a component, for example, one or more webs, of an absorbent article; providing a drum comprising a housing configured to rotate about a central longitudinal axis and defining an outer circumferential surface and an internal volume of the drum, fluid nozzles associated with the housing and receiving fluid from a fluid source, and fluid flow sensors associated with the housing, wherein the housing, the fluid nozzles, and the fluid flow sensors are configured to rotate about the central longitudinal axis; positioning the component of the absorbent article on the outer circumferential surface, wherein the component of the absorbent article is positioned such that fluid transmitted by one or more first fluid nozzles of the fluid nozzles contacts the component of the absorbent article; rotating the housing about the central longitudinal axis; transmitting a fluid from the fluid source to the one or more first fluid nozzles; monitoring a first fluid flow rate of the fluid transmitted to the one or more first fluid nozzles via a first fluid flow sensor of the fluid flow sensors as the one or more first fluid nozzles rotate about the central longitudinal axis; generating a first output corresponding to the first fluid flow rate via the first fluid flow sensor; transferring the first output to a data processing unit; monitoring via the data processing unit a first fluid flow rate of the fluid transmitted to the one or more first fluid nozzles using the first output. In one embodiment, the method is suitable for determining a fluid flow rate of a fluid in a drum for manufacturing absorbent articles as it rotates.

The method may further comprise: detecting an angular position of the one or more first fluid nozzles using an angular position sensor associated with the housing as the one or more first fluid nozzles rotate about the central longitudinal axis; transferring via the angular position sensor data corresponding to the angular position of the one or more first fluid nozzles as the one or more first fluid nozzles rotate about the central longitudinal axis; the monitoring comprises determining via the data processing unit the first fluid flow rate of the fluid transmitted to the one or more first fluid nozzles at each of a plurality of angular positions of the one or more first fluid nozzles that fall within a predefined angular data collection range.

The component of the absorbent article may comprise at least one of a first nonwoven, a second nonwoven, or an elastomeric material.

### BRIEF DESCRIPTION OF THE DRAWINGS

FIG. 1 is a schematic, cross-sectional view of an example of a radial pneumatic distribution system according to one or more embodiments shown and described herein;

FIG. 2 is a schematic, cross-sectional view of another example of a radial pneumatic distribution system according to one or more embodiments shown and described herein;

FIG. 3 is a schematic, cross-sectional view of a known radial pneumatic distribution system;

FIG. 4 is a schematic, side view of a portion of an example of a drum used in a radial pneumatic distribution system according to one or more embodiments shown and described herein;

FIG. 5 is a perspective view of an example of fluid nozzles suitable for use in a drum according to one or more embodiments shown and described herein;

FIG. 6 is a schematic, cross-sectional view of an example of a rotor suitable for use in a drum according to one or more embodiments shown and described herein;

FIG. 7A is a schematic, cross-sectional view of a rotor positioned about a dead shaft according to one or more embodiments shown and described herein;

FIG. 7B depicts an example of fluid flow through fluid pathways through the rotor of FIG. 7B at various times and states of rotation according to one or more embodiments shown and described herein;

FIG. 8A depicts fluid flow rate data of a fluid transmitted to a first fluid nozzle at a first seaming station and a second fluid nozzle at a second seaming station;

FIG. 8B illustrates only peak data of data curves C₁ and C₂ in FIG. 8A;

FIG. 9 illustrates graphs of Flow Average versus Time showing Target Flow compared to Leak Flow and Clogged Flow for a drum according to one or more embodiments shown and described herein;

FIG. 10A depicts a flowchart of an example process for collecting filtered data for a drum according to one or more embodiments shown and described herein;

FIG. 10B depicts a flowchart of an example process for collecting filtered data for a drum according to one or more embodiments shown and described herein;

FIG. 11 depicts a flowchart of example fluid pathways for a fluid through a drum according to one or more embodiments shown and described herein;

FIG. 12 depicts an example signal path of a sensor, for example, a fluid flow sensor, from a rotating side to a static side of a radial pneumatic distribution system according to one or more embodiments shown and described herein;

FIGS. 13A1-13C2 depict flowcharts of examples of methods useful for a radial pneumatic distribution system according to one or more embodiments shown and described herein;

FIGS. 14A1-14A3 depict flowcharts of examples of methods useful for a radial pneumatic distribution system according to one or more embodiments shown and described herein;

FIG. 15 illustrates a schematic side view of a portion of a radial pneumatic distribution system for making side seams on components of absorbent articles according to one embodiment shown and described herein; and

FIG. 16 is a perspective view of a diaper pant.

### DETAILED DESCRIPTION

In the following detailed description of the illustrated embodiments, reference is made to the accompanying drawings that form a part hereof, and in which is shown by way of illustration, and not by way of limitation. It is to be understood that other embodiments may be utilized and that changes may be made without departing from the spirit and scope of various embodiments illustrated herein.

The following term explanations may be useful in understanding the present disclosure:

“Absorbent article” is used herein to refer to consumer products whose primary function is to absorb and retain soils and wastes. “Diaper” is used herein to refer to an absorbent article generally worn by infants and incontinent persons about the lower torso. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

“Body-facing” or “wearer-facing” and “garment-facing” refer respectively to the relative location of an element or a surface of an element or group of elements. “Body-facing” or “wearer-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

“Disposed” refers to an element being located in a particular place or position.

“Proximal” refers to a portion being closer relative to the longitudinal center of an absorbent article, while “distal” refers to a portion being farther from the longitudinal center of the absorbent article.

“Water-permeable” and “water-impermeable” refer to the penetrability of materials in the context of the indented usage of disposable absorbent articles. Specifically, the term “water-permeable” refers to a layer or a layered structure having pores, openings, and/or interconnected void spaces that permit liquid water, urine, or synthetic urine to pass through its thickness in the absence of a forcing pressure. Conversely, the term “water-impermeable” refers to a layer or a layered structure through the thickness of which liquid water, urine, or synthetic urine cannot pass in the absence of a forcing pressure (aside from natural forces such as gravity). A layer or a layered structure that is water-impermeable according to this definition may be permeable to water vapor, i.e., may be “vapor-permeable”.

“Extendibility” and “extensible” mean that the width or length of the component in a relaxed state can be extended or increased.

“Elasticated” and “elasticized” mean that a component comprises at least a portion made of elastic material.

“Elongatable material”, “extensible material”, or “stretchable material” are used interchangeably and refer to a material that, upon application of a biasing force, can stretch to an elongated length of at least about 110% of its relaxed, original length (i.e. can stretch to 10 percent more than its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongatable material will be considered to be “elastic” or “elastomeric.” For example, an elastic material that has an initial length of 100 mm can extend at least to 150 mm, and upon removal of the force retracts to a length of at least 130 mm (i.e., exhibiting a 40% recovery). In the event the material recovers less than 40% of its elongation upon release of the applied force, the elongatable material will be considered to be “substantially non-elastic” or “substantially non-elastomeric”. For example, an elongatable material that has an initial length of 100 mm can extend at least to 150 mm, and upon removal of the force retracts to a length of at least 145 mm (i.e., exhibiting a 10% recovery).

“Radial” means a direction running from the center of a drum toward a drum's outer circumferential surface.

The term “substrate” and “web material” and “web” is used herein to describe a material which is primarily two-dimensional (i.e. in an XY plane) and whose thickness (in a Z direction) is relatively small (i.e. 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, rear waist fastened or seamed).

“Pre-fastened” refers herein to pant diapers manufactured and provided to consumers in a configuration wherein the front waist region and the back waist region are fastened or connected to each other as packaged, prior to being applied to the wearer. As such pant diapers may have a continuous perimeter waist opening and continuous perimeter leg openings designed for infant or adult wearers. As discussed in more detail below, a diaper pant can be pre-formed by various techniques including, but not limited to, joining together portions of the diaper using refastenable and/or permanent closure members (e.g., seams, heat bonds, pressure welds, adhesives, cohesive bonds, mechanical fasteners, etc.). In addition, pant diapers can be pre-formed anywhere along the circumference of the waist region (e.g., side fastened or connected, front waist fastened or connected, rear waist fastened or connected).

“Associated with” as used herein means two or more parts are in a working relationship with each other and/or are physically connected, attached, combined, and/or supported by one another. In one example, an angular position sensor may be associated with a drum in a non-physically connected, a non-physically attached, a non-physically combined, and/or a non-physically supported by relationship even though the angular position sensor can monitor and/or detect and/or identify an angular position of a part of the drum with respect to a dead shaft about which the drum and its part rotate, for example, an angular position of a fluid nozzle of the drum. In other words, the angular position sensor may not be attached to the drum or any part of the drum, but be in a working relationship position to monitor and/or detect the angular position of a fluid nozzle of the drum. Alternatively, the angular position sensor, may, like a fluid nozzle of the drum, be connected to and/or attached to and/or combined with and/or supported by the drum or a part of the drum such that the angular position sensor itself rotates about the central longitudinal axis as the drum rotates about it.

The present disclosure relates to radial pneumatic distribution systems, methods, and apparatuses, for example, drums, for manufacturing absorbent articles, and in particular, to radial pneumatic distribution systems, methods, and apparatuses, for example, drums, for bonding web materials and/or components, such as web materials, together to create seams, such as side seams, useful when assembling absorbent articles. As discussed below in more detail, the radial pneumatic distribution systems, methods, and apparatuses, for example, drums, herein may be configured to bond web materials and/or components, such as web materials, together between an apparatus, such as a rotating drum and an anvil, such as an anvil roll. The rotating apparatus, for example, a rotating drum, includes a fluid nozzle and optionally, a press member, wherein the press member, when present, may include a patterned surface that defines a length that extends in a cross direction. As such, a first web material and a second web material may be advanced in a machine direction onto the apparatus, such as a rotating drum. The first web material may be positioned between the second web material and the drum on a surface, for example, an outer circumferential surface of the drum. A fluid, such as air, which may be heated to a temperature sufficient to partially melt the first web material layer and/or ply and the second web material layer and/or ply is applied to the first web material layer and second web material layer through the first web material layer from a fluid nozzle. In one embodiment, as the drum rotates, a fluid may be transmitted through a fluid nozzle into the first and second web materials to partially melt a portion of the first and second web material layers. In another embodiment, as the drum rotates, the fluid nozzle may or may not move radially outward from a housing of the drum and directs and/or transmits a jet of the fluid into the first and second web materials to partially melt a portion of the first web material layer and a portion of the second web material layer. The fluid nozzle may then radially retract inward into the housing of the drum, and a press member, when present, may shift radially outward from the housing of the drum, wherein a length of the surface extends in the cross direction across the first web material and/or the second web material. The partially melted portion of the first web material layer and the partially melted portion of the second web material layer are then bonded together by being compressed between the surface of the housing of the drum and an anvil, for example, an anvil roll to create a seam and/or between the surface of the press member of the drum and an anvil, for example, an anvil roll to create a seam, such as a side seam in the web materials and/or component and/or ultimately the resulting absorbent article.

As discussed below, the apparatus, for example, the drum may be configured to partially melt and compress the web materials and/or components, for example, substrates used to make absorbent articles while they are traveling on the drum to minimize deformation to weak, partially melted substrates as the substrates advance in the machine direction (MD).

It is to be appreciated that although the radial pneumatic distribution systems, methods, and apparatuses, for example, drums, herein may be configured to bond and/or seam various types of web materials and/or components, for example, substrates, the radial pneumatic distribution systems, methods, and apparatuses, for example, drums, herein are discussed below in the context of manufacturing absorbent articles. In particular, the radial pneumatic distribution systems, methods, and apparatuses, for example, drums, are discussed in the context of bonding belt substrates together to form side seams of advancing, continuous lengths of absorbent articles during production. As discussed below, an advancing continuous length of absorbent articles may include a plurality of chassis connected with a continuous first belt substrate and a continuous second belt substrate. The continuous first and second belt substrates may be separated from each other along a cross-machine direction (CD) while advancing along the MD. Each chassis may extend in the CD and may include opposing first and second end regions separated by a central region, wherein the first end regions are connected with first belt substrate and the second end regions are connected with the second belt substrate. The chassis may also be spaced from each other along the MD. A folding apparatus may operate to fold the chassis around a folding axis along the central regions and to bring the second belt substrate and second end region of the chassis into a facing relationship with the first belt substrate and first end region of the chassis. In some embodiments, the first belt substrate, second belt substrate, and folded chassis advance in the MD onto the rotating drum's outer circumferential surface as described above. As the drum rotates, a fluid nozzle may direct and/or transmit a fluid, with or without moving radially outward, to and through an aperture in the outer circumferential surface of the drum. The fluid nozzle directs a jet of the fluid, for example, heated fluid through the aperture and onto an overlap area of the first and second belt substrates, which partially melts the overlap area. As the drum continues to rotate, the fluid nozzle may stop the flow of the fluid to and through the aperture and may retract radially inward from the aperture to reset if necessary. If a press member is present in the drum, the press member may move radially outward to and/or through the aperture to assist in compressing the overlap area. The partially melted overlap area is then compressed between the outer circumferential surface of the drum and/or press member and an anvil, for example, an anvil roll, creating a bond, for example, discrete bond sites, or seams between the first and second belt substrates. The drum may continue to rotate after the compression step and the press member, if present, may retract radially inward from the aperture. The continuous length of first and second belt substrates may be further advanced from the drum to a knife roll. The bonded, for example, seamed regions may then be cut by the knife roll along the CD to create a first side seam on an absorbent article and a second side seam on a subsequently advancing absorbent article.

### Radial Pneumatic Distribution System

The present disclosure provides a radial pneumatic distribution system utilized to create bonds, for example, seams such as side seams in web materials and/or components, for example, substrates for making absorbent articles and/or absorbent articles themselves.

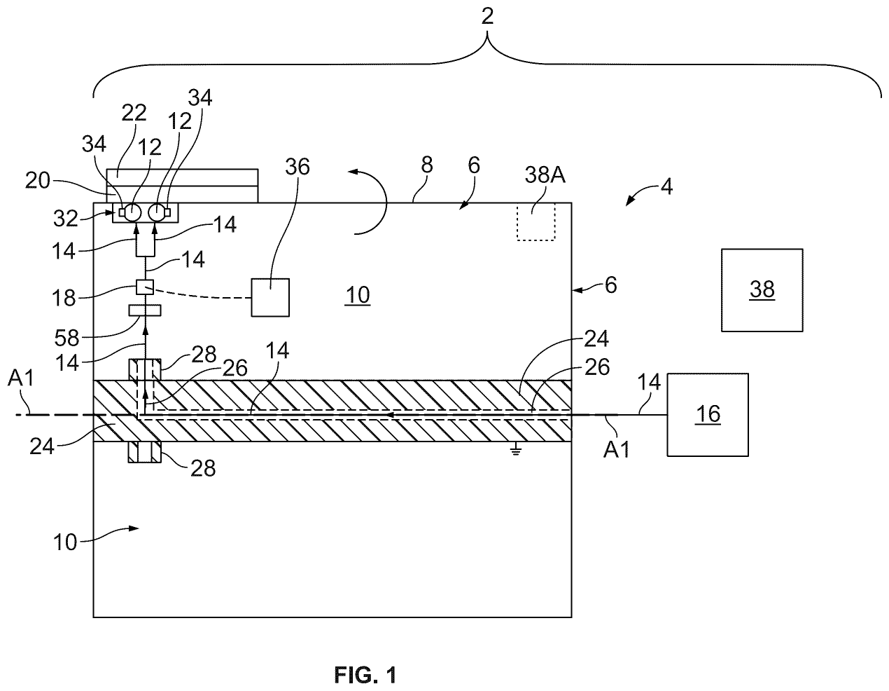

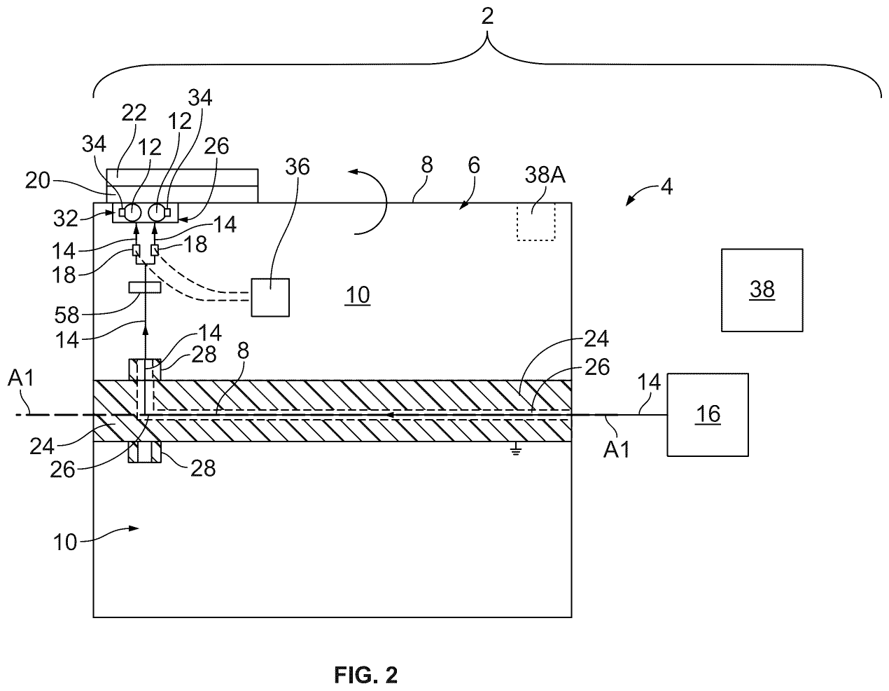

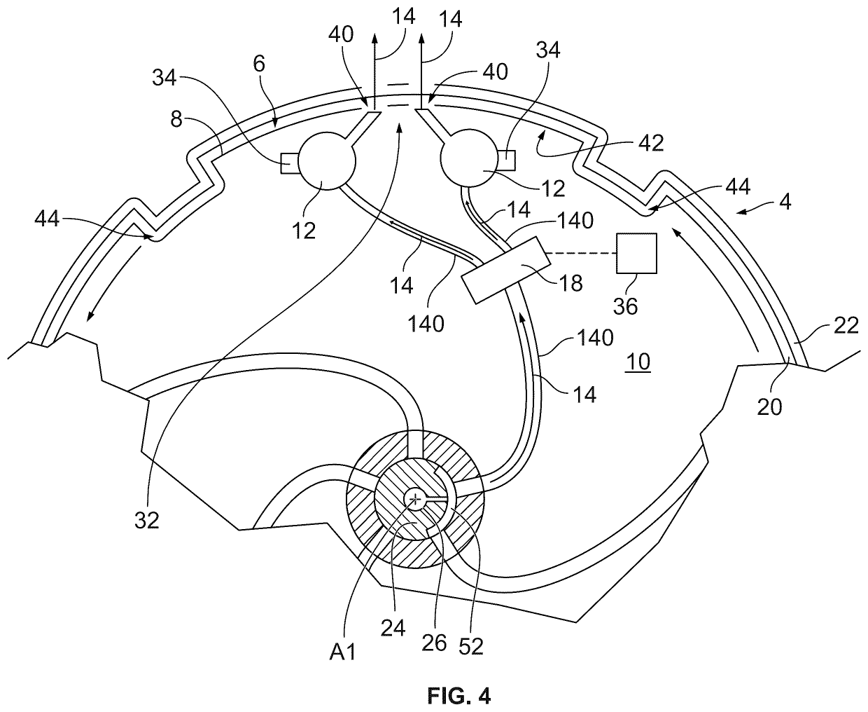

As shown in FIGS. 1 and 2 for purposes of illustration, and not by way of limitation, the radial pneumatic distribution system 2 of the present disclosure comprises an apparatus, for example, a drum 4 comprising a housing 6 configured to rotate about a central longitudinal axis A1 and defining an outer circumferential surface 8 and an internal volume 10 of the drum 4. The drum 4 further comprises fluid nozzles 12 associated with the housing 6 and receiving fluid 14 from a fluid source 16, and fluid flow sensors 18 associated with the housing 6, wherein the housing 6, the fluid nozzles 12, and the fluid flow sensors 18 are configured to rotate about the central longitudinal axis A1. In the example of FIG. 1, a pair of fluid nozzles 12 and a corresponding single fluid flow sensor 18 are shown located at a first circumferential location on the drum 4 near, i.e., slightly below, the outer surface 8 of the drum 4. One or more additional pairs of fluid nozzles 12 and a fluid flow sensor 18 corresponding to each pair of nozzles 12 may be provided on the drum 4, wherein the pairs of fluid nozzles 12 and corresponding fluid flow sensors 18 may be circumferentially spaced apart from one another on the drum 4. In one embodiment, the fluid source 16 may be fluidly connected to the fluid nozzles 12 for delivering the fluid 14, for example, a heated fluid (above 23° C. and/or above 32° C.), such as air, to a web, for example, two or more webs, such as a first web 20 and a second web 22, that are layered and/or plied together and that will ultimately form one or more component parts of an absorbent article. The fluid pathway of the fluid connection from the fluid source 16 to the fluid nozzles 12 may at least partially include a dead or static shaft 24 comprising a bore 26 through which the fluid 14 flows. The dead shaft 24 and bore 26 may be associated with a rotor 28 configured to radially distribute the fluid 14 to the fluid nozzles 12 along fluid pathways as the housing 6, the fluid nozzles 12, and the rotor 28 rotate about the central longitudinal axis A1 and the dead shaft 24. For example, fluid lines 140 may extend from the rotor 28 to the fluid flow sensors 18 and further fluid lines 140 may extend from the fluid flow sensors 18 to the nozzles 12, see FIG. 4, such that the fluid lines 140 may define the fluid pathways. In one embodiment, the fluid flow sensors 18 may be configured to monitor, detect, measure, and/or capture a fluid flow rate of the fluid 14 transmitted to and/or through the fluid nozzles 12. The rotor 28 is coupled to the drum 4 via a mechanical linkage (not shown) and is able to rotate relative to the dead shaft 24 with the drum 4 as the rotor 28 is coupled to the drum 4 via the mechanical linkage.

The radial pneumatic distribution system 2 is configured to provide fluid flow rate data, for example, peak, mean, median, and/or minimum fluid flow rate values, linked to one or more fluid nozzles 12 associated with the housing 6 as the drum 4 rotates about the central longitudinal axis A1 during operation. From such fluid flow rate data, a user, electronic processor and/or machine can determine if the radial pneumatic distribution system 2 and/or components thereof, for example, one or more fluid nozzles 12, are performing as intended and expected or if they are mis-performing. For example, if one or more of the fluid nozzles 12 is mis-performing, for example, if one or more of the fluid nozzles 12 or fluid pathways to the one or more fluid nozzles 12 is clogged or leaking, resulting seam or seams on the webs 20, 22 created by the one or more mis-performing fluid nozzles 12 may be too weak or too brittle or non-existent. With such information a user, electronic processor and/or machine may take action to adjust the fluid flow, e.g., rate of flow, to clean the fluid nozzles 12, to clean the fluid pathways, and/or to take other remedial actions including stopping the fluid flow of the fluid 14 and/or stopping operation of the radial pneumatic distribution system 2 including stopping the rotation of the housing 6 of the drum 4.

### Apparatus, for Example, Drum

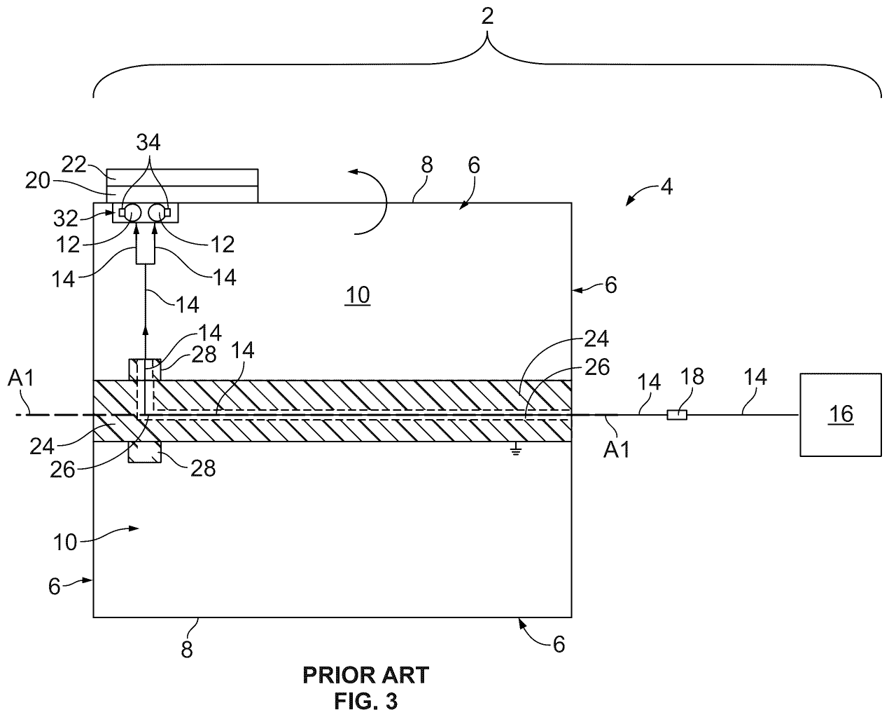

As shown in FIG. 3, known radial pneumatic distribution systems 2 fail to provide the ability to readily and accurately identify specific fluid nozzles 12 associated with the housing 6 of the drum 4, including their associated fluid pathways, that may be mis-performing, for example, clogged and/or leaking fluid, including percent clogged and/or percent leakage. In addition, the known radial pneumatic distribution systems 2 fail to provide the ability to identify the angular position of the specific fluid nozzles 12.

Even though the known radial pneumatic distribution systems 2 comprise a drum 4 configured to deliver a fluid 14, such as air, to webs, for example a first web 20 and a second web 22, that are, for example, in contact with one another in a face-to-face orientation, in order to create bonds, such as side seams on the webs and on the resulting absorbent articles containing the webs, the systems 2 do not include one or more fluid flow sensors located within or on the drum 4 to sense fluid flow at a corresponding one or a subset of the nozzles associated with the drum 4. As shown in FIG. 3, a fluid source 16, for example, an air source, for providing a fluid 14 into the drum 4 is positioned outside of the housing 6 of the drum 4 and is stationary relative to the housing 6 as the housing 6 rotates about the central longitudinal axis A1. The housing 6 of the drum 4 defines an outer circumferential surface 8 and an internal volume 10 of the drum 4. In operation, the fluid 14 enters the drum 4 from the fluid source 16 through a fluid pathway represented by an arrow. The fluid 14 passes through a fluid flow sensor 18, which is oftentimes referred to as a bulk fluid flow sensor, in fluid communication with the fluid source 16 and a dead shaft 24 having a bore 26. The dead shaft 24 is stationary relative to the housing 6 of the drum 4 as the housing 6 rotates about the central longitudinal axis A1. The dead shaft 24 extends into the drum 4 and is in fluid communication with a rotor 28 via the bore 26 of the dead shaft 24. After the fluid 14 enters the bore 26 of the dead shaft 24, the fluid 14 flows to the rotor 28. The rotor 28 rotates about the dead shaft 24 and/or the central longitudinal axis A1 as it distributes the fluid 14 to one or more fluid nozzles 12 and/or one or more groups of fluid nozzles 12 as the fluid nozzles 12 rotate about the central longitudinal axis A1. During operation of the drum 4 and radial pneumatic distribution system 2, the only location at which fluid flow rate data is monitored, detected, measured, and/or captured is at the fluid flow sensor 18, which is outside of the housing 6 of the drum 4. The fluid flow sensor 18 (bulk fluid flow sensor in this case) is positioned outside of the housing 6 of the drum 2 and is stationary and static relative to the housing 6 of the drum 4 as the housing 6 and the fluid nozzles 12 rotate about the central longitudinal axis A1. The location of the fluid flow sensor 18 outside of the drum 4 results in fluid flow rate data being monitored, detected, measured, and/or captured collectively for all of the fluid nozzles 12 within the drum 4 as a lump sum or bulk flow rate data collection without the ability to specifically monitor, detect, measure and/or capture the fluid flow rate of the fluid 14 to an individual fluid nozzle 12 or to an individual group of two or more fluid nozzles 12 in a seaming station 32 within the drum 4 as shown in FIG. 3 because the fluid flow sensor 18 is not specifically associated with or linked to an individual fluid nozzle 12 or a subset of fluid nozzles 12. As a result, the configuration of the known radial pneumatic distribution system 2 and drum 4 of FIG. 3 does not permit monitoring, detecting, measuring, and/or capturing fluid flow rate data nor angular position data for individual fluid nozzles 12 or subsets of fluid nozzles 12 and thus, does not permit the identification of clogs and/or leaks within individual fluid nozzles 12 and/or associated fluid pathways and/or within an individual group of two or more fluid nozzles 12 and/or associated fluid pathways within the drum 4. Thus, there is no ability to determine which fluid nozzle 12 or group of fluid nozzles 12 is malfunctioning or mis-performing, such as is clogged and/or is leaking fluid leading to potential defects in the bonds formed, for example, side seams formed, which may ultimately lead to defects in the absorbent articles produced therefrom.

As mentioned above, the negatives associated with the known radial pneumatic distribution system include the inability to readily and accurately identify specific fluid nozzles and/or fluid pathways delivering the fluid to the fluid nozzles that are mis-performing, for example, clogged and/or leaking including percent clogged and/or percent leakage, and angular positions of such mis-performing fluid nozzles and/or fluid pathways.

It has been found that a radial pneumatic distribution system 2 of the present disclosure including a drum 4 of the present disclosure overcomes the negatives associated with the prior art radial pneumatic distribution system and its drum 4 shown in FIG. 3 by providing the ability to readily and accurately identify specific fluid nozzles and/or fluid pathways that are mis-performing, for example, clogged and/or leaking including percent clogged and/or percent leakage, and angular positions of such mis-performing fluid nozzles and/or fluid pathways.

As shown in FIGS. 1, 2 and 4, an apparatus of the present disclosure, for example, a drum 4 configured to deliver a fluid 14, such as air, to one or more webs, for example, a first web 20 and a second web 22, in order to create bonds, such as side seams on the webs, and thus side seams in the resulting absorbent articles containing the webs, comprises a housing 6 that defines an outer circumferential surface 8 and an internal volume 10 of the drum 4. The fluid 14 may be a heated fluid, such as air, and/or may be a fluid 14 exhibiting a sufficient temperature to at least partially melt one or more of the first web and the second web 20, 22, such that a bond, for example, a side seam, may be formed on the webs.

The drum 4 of the present disclosure may comprise one or more seaming stations 32, wherein each seaming station 32 may comprise a fluid nozzle 12 or a group of two or more fluid nozzles 12 and optionally one or more heat exchangers 34. Each seaming station 32 may be positioned at a circumferential location on the drum 4 near, i.e., slightly below, the outer surface 8 of the drum 4. If a plurality of seaming stations 32 are provided, they may be circumferentially spaced apart from one another about the drum 4. A location on the outer circumferential surface 8 of the drum 4 near or at a given seaming station 32 is where the fluid 14 from the one or more of the fluid nozzles 12 of that seaming station contacts the webs, for example, the first web and the second web 20, 22, to soften and/or partially melt the webs.

As shown in FIGS. 1 and 4, the drum 4 of the present disclosure is configured to deliver a fluid 14, such as air, to a web, for example, a first web 20 and a second web 22, that are, for example, in contact with one another in a face-to-face orientation, in order to create bonds, such as side seams on the webs and on the resulting absorbent articles containing the webs. The drum 4 of the present disclosure via one or more fluid flow sensors 18 is capable of monitoring, detecting, measuring and/or capturing raw fluid flow rate data for the fluid 14 and transferring, directly or indirectly, via one or more data transfer units 36, which receive the raw fluid flow rate data from the one or more fluid flow sensors 18, to at least one data processing unit, which, in the example of FIGS. 1 and 2, may comprise a single data processing unit 38, which is static and not on the drum 4. It is also contemplated a further data processing unit 38A, shown in dotted line in FIGS. 1 and 2, may be provided on the drum 4. It is further contemplated that more than two data processing units may be provided. In one example, one or more data transfer units 36 are provided, such that each data transfer unit 36 receives raw fluid flow rate data or signals from two or more fluid flow sensors 18. For example, each data transfer unit 36 may be coupled to four fluid flow sensors 18 for collecting raw data from those four sensors 18. The data transfer units 36 may not process the raw data received from the fluid flow sensors 18 but collect the raw data from each associate fluid flow sensor 18 and transfer that raw data to the static processing unit 38. The data transfer units 36, when present, are associated with the housing 6, for example, coupled to the housing 6 within the internal volume 10 and/or coupled to the housing 6 on the outer circumferential surface 8 of the housing 6 of the drum 4. Non-limiting examples of suitable data transfer units 36 include I/O Link Master, Bluetooth communication sensors, Wifi-connected sensors, Analog to Digital converters, slip ring/unions, optocouplers. Each data transfer unit 36 receives raw analog or digital signals corresponding to fluid flow rate data from each of its corresponding one or more fluid flow sensors 18 and then transfers the raw analog or digital signals to the single, static data processing unit 38. The data transfer units 36 and fluid flow sensors 18 may form part of the rotating components (“rotating side”) of the radial pneumatic distribution system 2. The raw analog or digital signal from a fluid flow sensor 18 is also referred to herein as an output corresponding to a fluid flow rate as sensed by the fluid flow sensor.

If a data processing unit 38A is provided on the drum 4, the fluid flow sensors 18 are coupled directly to the data processing unit 38A such the processing unit 38A receives the raw signals from the fluid flow sensors 18 directly. The data processing unit 38A processes the raw signals to generate fluid flow rate values, where each fluid flow rate value is identified with the fluid flow sensor 18 that generated the raw data or signal from which the fluid flow rate value was calculated. Because the data processing unit 38A is provided on the drum 4 and is capable of receiving and processing the raw data quickly, the data processing unit 38A may time stamp each fluid flow rate value with a time corresponding to when the raw data corresponding to that fluid flow rate value was generated. The data processing unit 38A then transfers the fluid flow rate values to a data transfer unit (not shown) on the drum, which, in turn, transfers the fluid flow rate values to the stationary/static components (“static side”) of the radial pneumatic distribution system 2; namely, to the data processing unit 38 positioned separate from and outside of the housing 6 of the drum 4. The data processing unit 38 functions to analyze the fluid flow rate values to determine if a nozzle 12 is blocked or clogged or if a fluid flow leakage is occurring within the drum 4 and may generate an alert or corrective command to an operator or generate an electronic command causing the drum and possibly a production line, which includes the drum 4, to shut down.

If only a single data processing unit 38 is provided, it may perform the functions performed by the two data processing units 38, 38A discussed above, except that the single data processing unit 38 may not time stamp the calculated fluid flow rate data due to a lag in time in receiving the raw data from the drum 4.

The data processing unit 38 may be in communication with one or more of the fluid flow sensors 18 through a communication network as discussed above comprising the one or more data transfer units 36. As such, it is to be appreciated that the data processing unit 38 may be physically located near the drum 4 or may be located at another location and in communication with the fluid flow sensors 18 via a wired and/or wireless network. In some embodiments, the communication network is configured as a non-deterministic communication network, such as, for example, Ethernet or Ethernet IP (industrial protocol) communication network.

In one embodiment, a suitable data processing unit or units 38, 38A may include programmable logic controllers (PLC), PAC, CPU, Industrial PC, Intelligent Sensor, DAQ. In one embodiment, one or more of the fluid flow sensors 18 may have data processing capabilities and may include one or more of the above devices or associated functions suitable for data processing.

Further non-limiting examples of data processing units 38, 38A suitable for storing and/or executing program code may include at least one processor coupled directly or indirectly to memory elements, e.g., through a system bus or other suitable connection that can be used with the radial pneumatic distribution system 2 of the present disclosure and may include computer systems, for example, PLCs and/or personal computers (PCs) running software and adapted to communication on an EthernetIP network. The memory elements may include local memory employed during actual execution of the program code, memory that is integrated into a microcontroller or application specific integrated circuit (ASIC), a programmable gate array or other reconfigurable processing device, etc. The at least one processor may include any processing component operable to receive and execute executable instructions (such as program code from one or more memory elements). The at least one processor may comprise any kind of a device which receives input data, processes that data through computer instructions, and generates output data. Such a processor can be a microcontroller, a hand-held device, laptop or notebook computer, desktop computer, microcomputer, digital signal processor (DSP), mainframe, server, cell phone, personal digital assistant, other programmable computer devices, or any combination thereof. Such processors can also be implemented using programmable logic devices such as field programmable gate arrays (FPGAs) or, alternatively, realized as application specific integrated circuits (ASICs) or similar devices. The term “processor” is also intended to encompass a combination of two or more of the above recited devices, e.g., two or more microcontrollers.

Some data processing units 38, 38A may comprise or utilize industrial programmable controllers such as the Siemens S7 series, Rockwell ControlLogix, SLC or PLC 5 series, or Mitsubishi Q series. The aforementioned embodiments may use a personal computer or server running a control algorithm such as Rockwell SoftLogix or National Instruments Labview or may be any other device capable of receiving inputs from sensors, performing calculations based on such inputs and generating control actions through servomotor controls, electrical actuators or electro-pneumatic, electrohydraulic, and other actuators. In some configurations, the data processing unit(s) 38, 38A and/or components thereof may utilize a print quality management program wherein the system may upload quality data in a data center where a printer, color separator, and/or customer may view the data remotely and analyze the data for printing quality improvement. Examples of such print quality management programs are available from for example Schawk (ColorDrive), and X-rite (ColorCert). Process and product data may be stored directly in the aforementioned computer systems or may be located in a separate data historian. In some embodiments, the historian is a simple data table in the controller. In other embodiments, the historian may be a relational or simple database. Common historian applications include Rockwell Automation Factory Talk Historian, General Electric Proficy Historian, OSI PI, or any custom historian that may be configured from Oracle, SQL or any of a number of database applications. It is also to be appreciated that the data processing unit(s) 38, 38A may be configured to communicate with various types of controllers and inspection sensors configured in various ways and with various algorithms to provide various types of data and perform various functions.

In one embodiment, the fluid flow rate data for the fluid 14 flowing to one or more of the fluid nozzles 12 within the internal volume 10 of the housing 6 of the drum 4 as the housing 6 of the drum 4 rotates about the central longitudinal axis A1 is obtainable because one or more of the fluid flow sensors 18 are present within the internal volume 10 of the drum 4 and/or present on the outer circumferential surface 8 of the housing 6 of the drum 4 and rotate about the central longitudinal axis A1 as the housing 6 of the drum 4 rotates about the central longitudinal axis A1.

The drum 4 comprises a motor (not shown) that when powered drives and rotates the housing 6 of the drum 4 about the central longitudinal axis A1. The motor may comprise or be associated with an angular position sensor (not shown). In one embodiment, the motor comprises a servo motor that contains an angular position sensor, often times referred to as an internal motor resolver and/or a position transducer. The angular position sensor may generate encoder signals relative to a flag or known predefined point on the motor shaft passing the sensor, wherein the sensor is at a fixed reference location, such that the encoder signals correspond to an angular position of the motor shaft flag relative to the fixed reference location. The data processing unit(s) 38, 38A are provided with known positions of the one or more seaming stations 32 and, hence, the one or more corresponding fluid nozzles 12 at each seaming station 32, relative to the flag on the motor shaft, which positions of the seaming stations remain constant or fixed relative to the motor shaft flag and, accordingly, the data processing units 38, 38A are able to determine the angular location of each seaming station relative to the fixed reference location of the transducer as the drum 4 is turning. In one embodiment, an angular position sensor, for example, a position transducer or encoder, is associated with the drum 4 and dead shaft 24. The angular position sensor may generate encoder signals, i.e., angular position data, relative to a flag or known predefined point on the drum 4 passing the angular position sensor at a fixed reference location on the dead shaft 24, such that the encoder signals correspond to an angular position of the drum flag relative to the fixed reference location on the dead shaft 24. The encoder signals from the angular position sensor may be sent directly to the static data processing unit 38 when the angular position sensor is coupled or fixed to the fixed dead shaft 24 and the flag rotates with the housing. When the rotating data processing unit 38A is provided on the drum, the encoder signals from the angular position sensor coupled to the fixed dead shaft 24 may be sent first to the data transfer unit 36, which then forwards the angular position data to the rotating data processing unit 38A. The data processing unit(s) 38, 38A are provided with known positions of the one or more seaming stations 32 and, hence, the one or more corresponding fluid nozzles 12 at each seaming station 32, relative to the flag on the drum, which positions of the seaming stations remain constant or fixed relative to the drum flag and, accordingly, the data processing units 38, 38A are able to determine the angular location of each seaming station relative to the fixed reference location on the dead shaft 24 as the drum 4 is turning. In one embodiment, an angular position sensor, for example, an external position transducer may be present on the motor, for example, an AC motor. The angular position sensor and/or position transducer may comprise a position encoder, resolver, and/or an inductive positioning device. Non-limiting examples of position encoders include Sick Hiperface and EnDat, both commercially available. The position encoder may also utilize an optical pulse with TTL signal.

The angular position sensor, directly or indirectly via a data transfer unit 36, provides angular position data to the data processing unit(s) 38, 38A. The data processing unit(s) 38, 38A can utilize the angular position data and the fluid flow rate data or values to identify which fluid nozzle 12 or fluid nozzles 12 around the circumference, for example, near the outer circumferential surface 8 of the housing 6 of the drum 4, are exhibiting a condition, such as reduced or no fluid flow condition, that needs to be addressed. If the data processing unit 38A is positioned within the internal volume 10 of the housing 6 of the drum 4, the data processing unit 38A may then send the information to the data processing unit 38 positioned separate from and outside of the housing 6 of the drum 4 and is stationary relative to the housing 6 as the housing 6 of the drum 4 rotates about the central longitudinal axis A1 during operation.

The data processing unit(s) 38, 38A may, in response to the processed fluid flow rate data or calculated fluid flow rate values and the angular position data, send an alert, command or otherwise signal that an issue with one or more fluid nozzles 12 and/or fluid pathways associated with the one or more fluid nozzles 12 needs to be addressed. To address the issue, such as a clogged fluid nozzle or associated fluid pathway or a leaking fluid nozzle or associated fluid pathway, an increase or decrease in fluid flow to the fluid nozzle 12 or fluid nozzles 12 may be effected by a flow control or pressure control valve 58, such as a solenoid activated valve. Other actions that may be taken include stopping the flow of the fluid 14 before going into the drum 4 or only at one or a subset of two or more nozzles 12 via a corresponding flow control valve 58, stopping the rotation of the housing 6 of drum 4, issuing an alert to a user, and/or any of such actions resulting in stopping, at least temporarily, the production of the absorbent articles so that the issue can be addressed. For example, the data processing device 38, 38A may generate a flow control signal to the flow or pressure control valve 58 to change a size of an opening of the valve 58 so as to increase, decrease or stop the flow of fluid moving through one or more pathways downstream from the valve 58. Hence, the fluid flow through the nozzle or two or more nozzles located downstream of the valve 58 will be increased, decreased or stopped by the action of the valve 58. As shown in FIG. 1, one valve 58 is provided in or before a seaming station 32 between the rotor 28 and a single nozzle 12. As shown in FIG. 2, one valve 58 is provided in or before a seaming station 32 between the rotor 28 and a pair of nozzles 12. While a single control valve 58 is provided in or before each of seaming station 32 in FIGS. 1 and 2, it is contemplated that two or more valves 58 may be provided in or before a seaming station 32. The data processing unit(s) 38, 38A may utilize a closed loop system to address the issues with the fluid flow rate within the drum 4. The flow control or pressure control valve 58 may be a proportion valve allowing incremental or variable flow control adjustments or an ON/OFF or binary valve allowing only fully on or fully off flow control.

In one embodiment, as shown in FIGS. 1 and 4, during operation of the drum 4 and the radial pneumatic distribution system 2, the fluid source 16, for example, an air source, provides a fluid 14 into the drum 4 to the fluid nozzles 12. The fluid source 16 is positioned separate from and outside of the housing 6 of the drum 4 and is stationary relative to the housing 6 as the housing 6 of the drum 4 rotates about the central longitudinal axis A1. In one embodiment, the fluid 14 enters the drum 4 from the fluid source 16 through the fluid passageway formed by the bore 26 of the dead shaft 24. The dead shaft 24 is stationary relative to the housing 6 as the housing 6 of the drum 4 rotates about the central longitudinal axis A1. The dead shaft 24 extends into the drum 4 and is in fluid communication with the rotor 28 via the bore 26 of the dead shaft 24. After the fluid 14 enters the bore 26 of the dead shaft 24, the fluid 14 passes to the rotor 28. The rotor 28 rotates about the dead shaft 24 and/or the central longitudinal axis A1 as it distributes the fluid 14 to one or more fluid nozzles 12 and/or one or more groups of fluid nozzles 12 as the fluid nozzles 12 rotate about the central longitudinal axis A1. During operation of the drum 4 and the radial pneumatic distribution system 2, fluid flow rate data is monitored, detected, measured, and/or captured by the one or more fluid flow sensors 18. The one or more fluid flow sensors 18, which are in fluid communication with the one or more fluid nozzles 12 and the rotor 28, are positioned inside the drum 4 and rotate about the central longitudinal axis A1 as the housing 6 of the drum 4, fluid nozzles 12 and rotor 28 rotate about the central longitudinal axis A1.

As shown in FIG. 1, the drum 4 is configured to deliver the fluid 14 to the webs 20, 22, such as web materials and/or component parts of absorbent articles and/or absorbent articles in order to create bonds, such as side seams on the webs 20, 22, and ultimately the resulting absorbent articles.

In one embodiment, as shown in FIG. 2, operation of the drum 4 is similar to that shown and described in FIGS. 1 and 4, except that the fluid flow rate data is monitored, detected, measured, and/or captured by two fluid flow sensors 18 at the one seaming station 32 illustrated. Each seaming station 32 of the drum 4 may also comprise two fluid flow sensors 18. In this case, each fluid flow sensor 18 is in fluid communication with a single fluid nozzle 12 such that specific fluid flow rate data for the fluid 14 flowing to the single fluid nozzle 12 can be monitored, detected, measured, and/or captured during operation of the drum 4 and the radial pneumatic distribution system 2. The fluid flow sensors 18, which are each in fluid communication with a single fluid nozzle 12, are positioned inside the internal volume 10 of the drum 4 and rotate about the central longitudinal axis A1 as the housing 6 of the drum 4, fluid nozzles 12 and rotor 28 rotate about the central longitudinal axis A1. Further, the fluid source 16 is stationary relative to the fluid flow sensors 18, which rotate about the central longitudinal axis A1 during operation.

As shown in FIG. 4, the drum 4 further comprises one or more apertures 40 within its housing 6 around its outer circumferential surface 8. At least one of the apertures 40 is associated with at least one of the fluid nozzles 12 such that fluid 14 may pass from the at least one fluid nozzle 12 to outside the drum 4 through the aperture 40 and contact the webs 20, 22.

In one embodiment, the drum 4 may include a plurality of fluid nozzles 12 present within the internal volume 10 of the housing 6 of the drum 4 and in fluid communication with at least one aperture 40. The plurality of fluid nozzles 12 may be associated with an inner circumferential surface 42 of the housing 6 of the drum 4 and be located at and/or affixed to a plurality of positions about the inner circumferential surface 42. In one embodiment, the drum 4 comprises a plurality of apertures 40 that are spaced about the outer circumferential surface 8 of the housing 6 of the drum 4. As shown in FIG. 4, the apertures 40 may be in groups of two or more apertures 40. For example, one group, such as a pair of apertures 40, may be associated with a group of fluid nozzles 12, such as a pair of fluid nozzles 12, and/or may be associated with a single seaming station 32. In another embodiment, the drum 4 comprises 2 or more, 3 or more, 4 or more, at least 5 and/or at least 6 apertures 40 and/or groups of apertures 40, such as pairs of apertures 40, spaced, for example, equally spaced, about the outer circumferential surface 8 of the housing 6 of the drum 4. A group of two or more apertures 40 along with their associated fluid nozzles 12 may be referred to as the seaming station 32 and/or a bonding station in FIG. 4. Each aperture 40 may be in fluid communication with an associated fluid nozzle 12 and/or group of fluid nozzles 12. As noted above, a plurality of seaming stations 32 may be spaced apart circumferentially about the drum 4, such that each seaming station 32 may comprise a pair of apertures 40 and a pair of fluid nozzles 12.

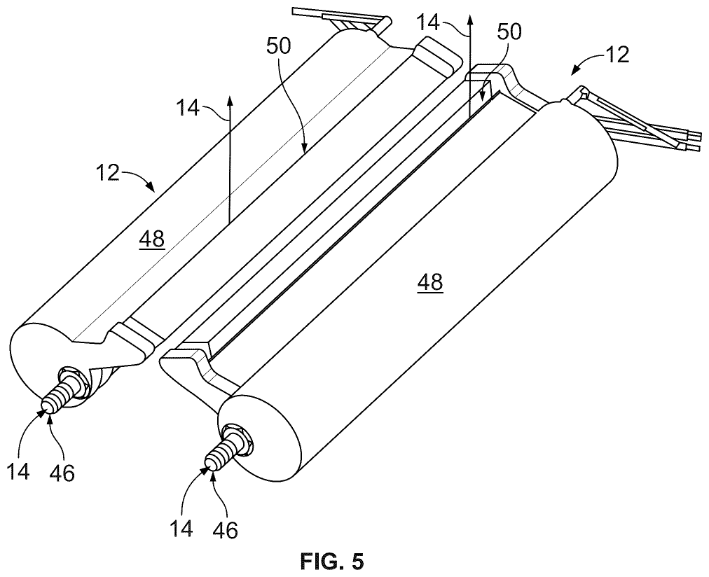

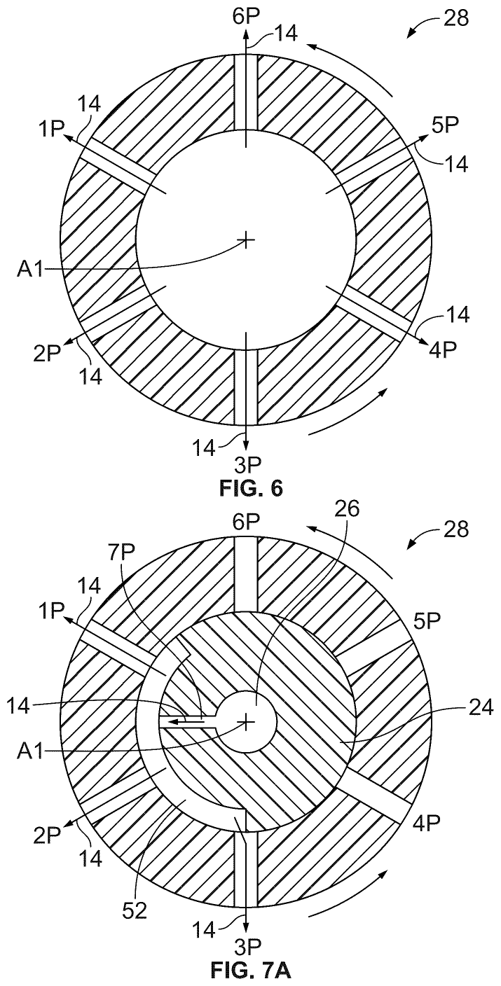

FIG. 5 shows an example of suitable fluid nozzles 12 for use in the drum 4. Each fluid nozzle 12 shown in FIG. 5 has an entrance 46 through which a fluid 14 enters a body 48 of the fluid nozzle 12 and an exit 50 through which the fluid 14 exits the body 48 as represented by the arrows. The entrance 46 is in fluid communication with a fluid pathway between the fluid source 16 and the fluid nozzle 12 as shown in FIGS. 1, 2, and 4. A single fluid flow sensor 18 as shown in FIG. 1 may be associated with each of the two fluid nozzles 12 and may be in fluid communication with the fluid pathway that communicates with the two fluid nozzles 12. The fluid flow sensor 18 in the FIG. 1 example senses the fluid flowing to the two fluid nozzles 12, i.e., at a point before the two nozzles. If the two nozzles 12 in the FIG. 1 example are working properly, the flow rate through each nozzle may be less than the flow rate detected by the fluid flow sensor 18 since the nozzles 12 are located downstream from the sensor 18. In another embodiment, the two fluid flow sensors 18 as shown in FIG. 2 may each, individually, be associated with a single fluid nozzle 12 such that a single seaming station 32 comprises two fluid nozzles 12 each of which is associated with a single fluid flow sensor 18. In the FIG. 2 example, if the two nozzles are working properly, the flow rate sensed by each fluid flow sensor 18 may be generally equal to the flow rate through its corresponding nozzle. In another embodiment, the drum 4 may comprise one seaming station 32 comprising two fluid nozzles 12 each of which is associated with a single fluid flow sensor 18 and another seaming station 32 comprising two fluid nozzles 12 that are associated with a single fluid flow sensor 18.

In one embodiment, the drum 4 may comprise two or more groups of one or more fluid nozzles 12, wherein each group of fluid nozzles 12 comprises one or more fluid nozzles 12 supported by the housing 6 of the drum 4. The two or more groups of fluid nozzles 12 may be spaced circumferentially apart from one another about and near the outer circumferential surface 8 of the housing 6 of the drum 4.

In one embodiment, the drum 4 may comprise a plurality of fluid flow sensors 18 which are grouped into two or more groups of one or more fluid flow sensors 18, wherein each group of fluid flow sensors 18 is associated with a corresponding group of one or more fluid nozzles 12.

In another embodiment, the drum 4 may comprise at least one group of two or more fluid nozzles 12 comprising a first fluid nozzle 12 and a second fluid nozzle 12 associated with a fluid flow sensor 18 for monitoring, detecting, measuring, and/or capturing fluid flow of a fluid 14 to the first and second fluid nozzles 12.

In still another embodiment, the drum 4 may comprise at least one group of two or more fluid nozzles 12 comprising a first fluid nozzle 12 and a second fluid nozzle 12 and a group of two or more fluid flow sensors 18 comprising a first fluid flow sensor 18 and a second fluid flow sensor 18 wherein the first fluid flow sensor 18 monitors, detects, measures, and/or captures the fluid flow rate of a fluid 14 to the first fluid nozzle 12 and the second fluid flow sensor 18 monitors, detects, measures, and/or captures the fluid flow of a fluid 14 to the second fluid nozzle 12.

In one embodiment, the plurality of fluid nozzles 12 may comprise two or more groups of fluid nozzles 12 supported by the housing 6, wherein the two or more groups of fluid nozzles 12 are spaced apart from one another about the housing 6 and wherein each of the two or more groups of fluid nozzles 12 is associated with one of the plurality of apertures 40. Further, the plurality of fluid flow sensors 18 may comprise two or more groups of fluid flow sensors 18, each of the two or more groups of fluid flow sensors 18 being associated with a corresponding group of fluid nozzles 12.

In one embodiment, the plurality of fluid nozzles 12 may comprise a first group of fluid nozzles 12 comprising a first fluid nozzle 12 and a second fluid nozzle 12, wherein one of the plurality of fluid flow sensors 18 is associated with the first fluid nozzle 12 and the second fluid nozzle 12 and is configured to detect a fluid flow rate of a fluid flowing to the first fluid nozzle 12 and to the second fluid nozzle 12.

In one embodiment, the plurality of fluid nozzles 12 may comprise a first group of fluid nozzles 12 comprising a first fluid nozzle 12 and a second fluid nozzle 12, wherein the plurality of fluid flow sensors 18 comprises a first fluid flow sensor 18 associated with the first fluid nozzle 12 and configured to detect a fluid flow rate of a fluid flowing to the first fluid nozzle 12 and a second fluid flow sensor 18 associated with the second fluid nozzle 12 and configured to detect a fluid flow rate of a fluid flowing to the second fluid nozzle 12.

In one embodiment, two or more fluid nozzles 12 of the plurality of fluid nozzles 12 may be present within the internal volume 10, wherein a first fluid nozzle 12 of the two or more fluid nozzles 12 being in fluid communication with a first fluid flow sensor 18 and a second fluid nozzle 12 of the two or more fluid nozzles 12 being in fluid communication with a second fluid flow sensor 18.

In one embodiment, a first fluid flow sensor 18 may monitor, detect, measure, and/or capture flow rate data regarding a fluid flowing to a first fluid nozzle 12 and a second fluid flow sensor 18 may monitor, detect, measure, and/or capture flow rate data regarding a fluid flowing to a second fluid nozzle 12 during operation of the drum 4 and the radial pneumatic distribution system 2. The first fluid flow sensor 18 may detect, measure, and/or capture a change in the fluid flow rate to the first fluid nozzle 12 and the second fluid flow sensor 18 may detect, measure, and/or capture a change in the fluid flow rate to the second fluid nozzle 12 during operation of the drum 4 and the radial pneumatic distribution system 2. The first and second fluid nozzles 12 may be associated with, for example, coupled to the inner circumferential surface 42 of the housing 6 of the drum 4.

In one embodiment, the outer circumferential surface 8 of the housing 6 may comprise one or more apertures 40 through which a fluid 14 delivered from one or more fluid nozzles 12 exits the internal volume 10 of the housing 6 of the drum 4 and passes at least into one or more of the webs 20, 22, as the housing 6 rotates during operation.

In one embodiment, the outer circumferential surface 8 of the housing 6 may comprise one or more apertures 40 through which a fluid 14 enters the internal volume 10 of the housing 6 of the drum 4 after passing through the one or more webs 20, 22, residing on the outer circumferential surface 8 of the housing 6 as the housing 6 rotates during operation.

In one embodiment, the outer circumferential surface 8 of the housing 6 may comprise one or more apertures 40 through which a fluid 14 delivered from one or more fluid nozzles 12 exits the internal volume 10 of the housing 6 of the drum 4 and passes at least into one or more of the webs 20, 22, as the housing 6 rotates during operation and the outer circumferential surface 8 of the housing 6 may comprise one or more apertures 40 through which a fluid 14 enters the internal volume 10 of the housing 6 of the drum 4 after passing through the one or more webs 20, 22, residing on the outer circumferential surface 8 of the housing 6 as the housing 6 rotates during operation.

In one embodiment, at least one of the plurality of fluid flow sensors 18 revolves around the central longitudinal axis A1 of the drum 4 during operation and rotation of the housing 6 of the drum 4.

At least one of the plurality of fluid flow sensors 18 may be in fluid communication with a pathway in fluid communication with a fluid source 16. The fluid source 16 may be positioned outside the outer circumferential surface 8 of the housing 6 of the drum 4. The fluid source 16 may be a stationary fluid source 16 relative to at least one fluid flow sensor 18 and/or at least one fluid nozzle 12 during rotation of the housing 6 of the drum 4 about the central longitudinal axis A1.

In one embodiment, a first portion of a plurality of fluid flow sensors 18, but less than all of the plurality of fluid flow sensors 18 monitors, detects, measures, and/or captures fluid flow to a first portion of a plurality of fluid nozzles 12, but less than all of the plurality of fluid nozzles 12 cyclically during operation and rotation of the housing 6 of the drum 4.

One or more of the fluid nozzles 12 within the internal volume 10 of the drum 4 may be radially positioned from the rotor 28 towards the inner circumferential surface 42 of the housing 6 of the drum 4.

The fluid flow sensors 18 of the present disclosure are configured to monitor, detect, measure, and/or capture, for example an increase or decrease, in the flow of a fluid 14 transmitted to a fluid nozzle 12 during rotation of the housing 6 of the drum 4.

In one embodiment, one or more fluid nozzles 12 may be in fluid communication with a heat exchanger 34. When the drum 4 comprises two or more fluid nozzles 12, a first fluid nozzle 12 may be in fluid communication with a first heat exchanger 34 and a second fluid nozzle 12 may be in fluid communication with a second heat exchanger 34 different from the first heat exchanger 34.

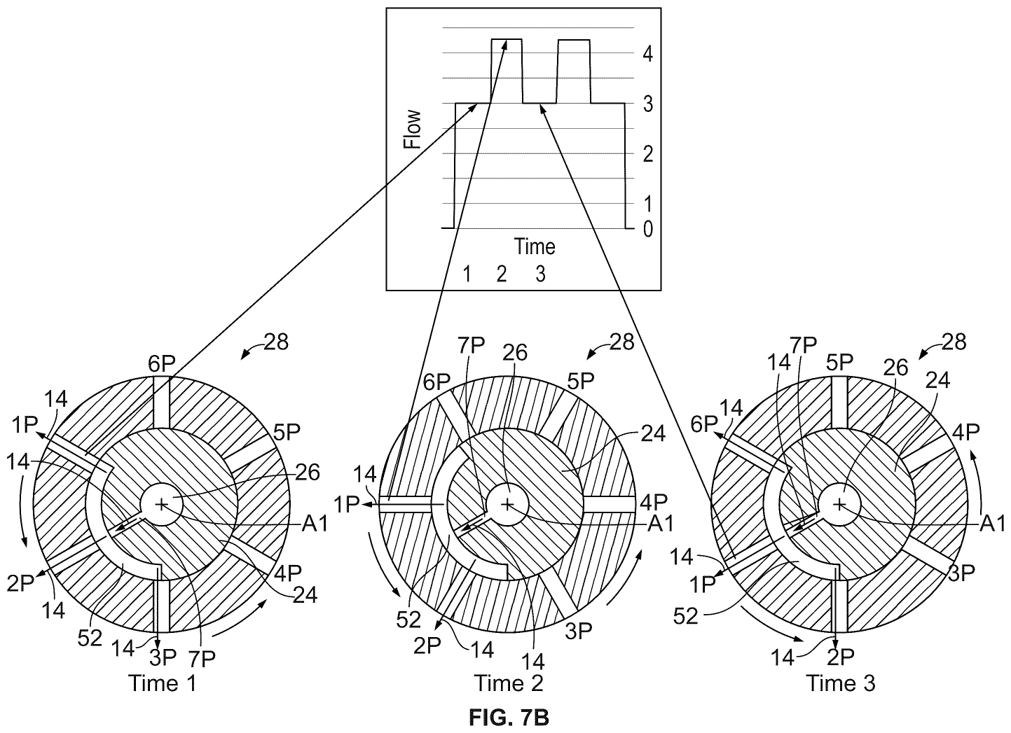

In one embodiment as described above, fluid 14 passes through the fluid nozzles 12 in the drum 4. As shown in FIG. 6, an example of a rotor 28 suitable for use in the drum 4 for managing the flow of the fluid 14 comprises one or more, in this case, a plurality of fluid pathways 1P-6P through which a fluid 14 is radially distributed from the rotor 28 to one or more fluid nozzles 12. The fluid flow of the fluid 14 from the rotor 28 may be regulated or controlled, such that only certain fluid pathways 1P-6P have the fluid 14 flowing through them at certain angular positions relative to the central longitudinal axis A1 and/or relative to a fixed reference location on the dead shaft 24 as the rotor 28 rotates about the central longitudinal axis A1, or unregulated such that all of the fluid pathways 1P-6P have the fluid 14 flowing through them at all times as the rotor 28 rotates about the central longitudinal axis A1. As noted above, the fluid flow of the fluid 14 can be regulated or controlled downstream from the rotor 28 via one or more flow or pressure control valves 58 provided prior to or in each seaming station 32, wherein each flow or pressure control valve 58 may be solenoid actuated or otherwise include an electronic actuator and controlled by the data processing unit(s) 38, 38A via flow control signals generated by the data processing unit(s) 38, 38A, such that a flow or pressure control valve 58 may turn fluid flow on and off at corresponding fluid nozzles 12 at pre-determined times and/or angular positions of rotation of the rotor 28. A manifold, which may be provided in the dead shaft 24, discussed further below, may allow delivery of flow of the fluid 14 to different fluid pathways 1P-6P and, hence, to different fluid nozzles 12 at pre-determined times and/or angular positions of rotation of the rotor 28. When an unregulated rotor 28, without any flow or pressure control valves or a manifold, is utilized, the fluid flow sensors 18 associated with the fluid nozzles 12 may monitor, detect, measure, and/or capture the fluid flow rate data associated with all of the fluid pathways 1P-6P continuously. When a regulated rotor 28 is utilized, the fluid flow rate data associated with one or more certain fluid pathways 1P-6P can be identified and the position of associated fluid nozzles 12 associated with the fluid pathways based on the angular position data can be used to determine the position of the fluid nozzle 12 or group of fluid nozzles 12 that are malfunctioning or mis-performing. This regulated scenario is similar to the situation described below in FIGS. 7A and 7B for a rotor 28 used in combination with a manifold 52, which manifold 52 may form part of the dead shaft 24. The manifold 52 is formed within and is stationary with the dead shaft 24. In FIGS. 7A and 7B, the dead shaft 24 is shown in cross section.

In FIG. 7A, the manifold 52 can be used with the rotor 28 to regulate the fluid flow of the fluid 14 through certain fluid pathways as the rotor 28 rotates about the central longitudinal axis A1 and the dead shaft 24. In the state of revolution shown in FIG. 7A, only fluid pathways 1P-3P communicate with the manifold 52 and receive fluid from the manifold 52 such that only fluid enters and exits the fluid pathways 1P-3P. The manifold 52 is stationary relative to the rotor 28 as the rotor 28 rotates about the central longitudinal axis A1 and the dead shaft 24 and its corresponding bore 26. The fluid 14 passes through the bore 26 of the dead shaft 24 to the manifold 52 through one or more fluid pathways, one such fluid pathway 7P is shown in FIGS. 7A and 7B. As the rotor 28 further rotates from the state shown in FIG. 7A, as represented by the arrow in FIG. 7A, fluid pathway 3P will move out of range of the manifold 52 and then only fluid pathways 1P and 2P will have fluid flow of the fluid 14 passing through them. Fluid flow rate data associated with 2-3 fluid pathways during the state illustrated in FIG. 7A and a short time thereafter, for example, fluid pathways 1P-3P or fluid pathways 1P and 2P, can be identified and the position of associated fluid nozzles 12 associated with the fluid pathways based on the angular position data can be used to determine the position of and the fluid nozzle 12 or group of fluid nozzles 12 that may be malfunctioning or mis-performing.

FIG. 7B illustrates the fluid flow of a fluid 14 through two or three fluid pathways over a period of time as the rotor 28 of FIG. 7A rotates about the dead shaft 24, the bore 26, the manifold 52 and the central longitudinal axis A1. As shown in FIG. 7B, the manifold 52 only permits the fluid 14 to pass through the fluid pathways 1P-3P in Time 1 state. No fluid 14 is passing through the fluid pathways 4P-6P in Time 1 state. The fluid flow of the fluid 14 in Time 1 state is distributed between three fluid pathways 1P-3P, such that the fluid flow through fluid pathway 1P comprises about 3 units as represented in the graph of FIG. 7B (the fluid flow through pathways 2P and 3P is not included in the graph of FIG. 7B). As the rotor 28 rotates to the state at Time 2, the fluid pathway 3P rotates past the manifold 52 and thus the fluid 14 ceases passing through fluid pathway 3P at that time as shown in FIG. 7B. The fluid 14 won't pass through the fluid pathway 3P again until the rotor 28 rotates sufficiently for the fluid pathway 3P to engage the manifold 52 again. Once the fluid pathway 3P passes the manifold 52, the fluid flow of the fluid 14 only flows through the fluid pathways 1P and 2P as shown in Time 2 state. The fluid flow of the fluid 14 in Time 2 state is distributed between only two fluid pathways 1P and 2P, such that the amount of fluid flowing through fluid pathway 1P is now about 4.5 units, as represented in the graph of FIG. 7B, which is greater than the 3 units at Time 1. This condition continues until the rotor 28 sufficiently rotates to permit the fluid pathway 6P to engage the manifold 52 as shown in Time 3 state in FIG. 7B. In Time 3 state, the fluid 14 passes through three fluid pathways again; namely, fluid pathways 1P, 2P and 6P. The fluid flow of the fluid 14 in Time 3 state is distributed between three fluid pathways 1P, 2P and 6P, such that the amount of fluid flowing through fluid pathway 1P is again about 3 units, as represented in the graph of FIG. 7B. As the rotor 28 continues to rotate about the central longitudinal axis A1, the rotor 28 passes through similar Time states having three fluid pathways active and then two fluid pathways active and then three fluid pathways active and two fluid pathways active and so on for the different fluid pathways 1P-6P of the rotor 28.

As described herein, once the fluid 14 passes through one or more of the fluid pathways 1P-6P of the rotor 28, the fluid 14 continues through fluid pathways, as sensed by one or more fluid flow sensors 18 and further to one or more fluid nozzles 12 and then ultimately exiting the housing 6 of the drum 4 via one or more apertures 40 in the outer circumferential surface 8 of the housing 6 of the drum 2, such as at a seaming station 32.

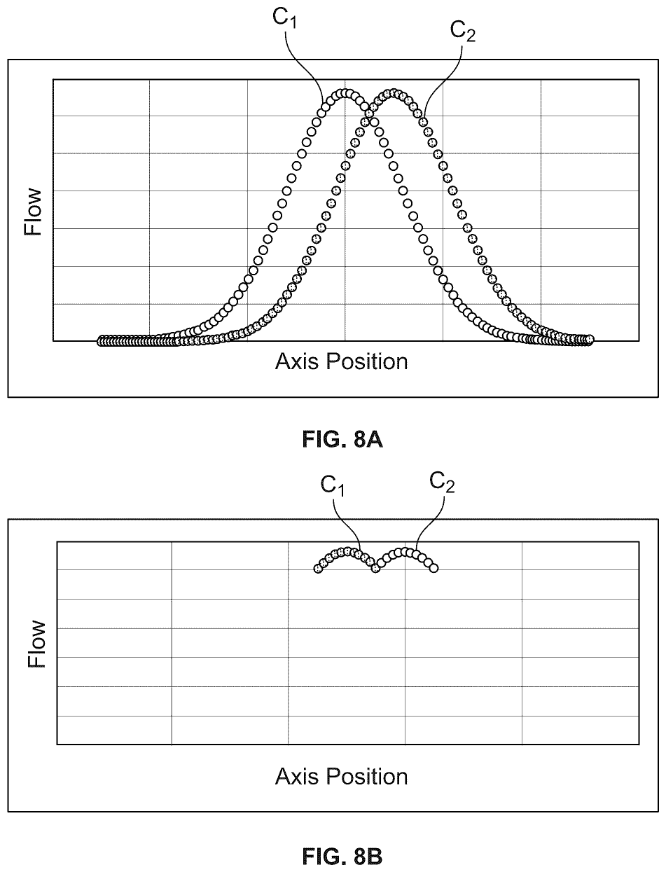

The chart of FIG. 8A depicts fluid flow rate data of a fluid 14 transmitted to a first fluid nozzle 12 at a first seaming station 32 and a second fluid nozzle 12 at a second seaming station positioned on the drum 4 at a circumferential location different from the first seaming station 32. More specifically, FIG. 8A illustrates a first curve C₁ corresponding to a fluid flow rate through the first fluid nozzle 12 over one full rotation of the drum 4, the rotor 28 and the first nozzle and a second curve C₂ corresponding to a fluid flow rate through the second fluid nozzle 12 over one full rotation of the drum 4, the rotor 28 and the second nozzle. The flow rate data to monitor fluid flow through a corresponding one or more nozzles may be limited to the peak fluid flow data from the corresponding fluid flow sensor 18. Hence, to reduce data processing and memory storage requirements, the fluid flow sensor data from the fluid flow sensors 18 captured, stored and/or monitored may only be the fluid flow data within a peak window as shown in FIG. 8B, i.e., only the peak data of the curves C₁ and C₂. Capturing, storing and/or monitoring only peak fluid flow data is referred to herein as filtering fluid flow rate data.

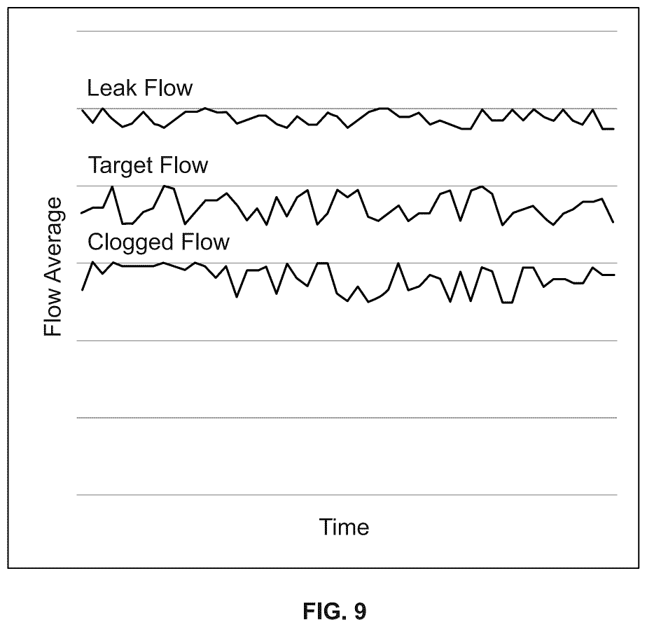

FIG. 9 depicts examples of fluid flow rate moving average data vs. time for a fluid flow of a fluid 14 through a single fluid flow sensor 18 and passing through one or more fluid nozzles 12 located downstream from the fluid flow sensor 18, wherein the single fluid flow sensor 18 and the one or more corresponding nozzles 12 may be in a single seaming station 32. When there is a leak through one of the nozzles downstream from the fluid flow sensor 18, at least one value of the fluid flow rate moving average may be higher than an upper value of an expected fluid flow rate moving average range, see FIG. 9. When there is a clogged nozzle 12 downstream from the fluid flow sensor 18, at least one value of the fluid flow moving average may be lower than a lower value of the expected fluid flow rate moving average range, see FIG. 9. In one example, there is an expected or normal fluid flow rate moving average range having upper and lower warning threshold values as well as upper and lower stop threshold values. The upper threshold stop value may be greater than the upper warning threshold value. The lower threshold stop value may be less than the lower warning threshold value. If one value of the fluid flow rate moving average exceeds the upper warning threshold value, then a leak alert may be generated to the operator and/or a corrective command may be generated by the data processing unit 38, 38A. If one value of the fluid flow rate moving average is lower than the lower warning threshold value, then a clog alert may be generated to the operator and/or a corrective command may be generated by the data processing unit 38, 38A, such as a flow control signal being generated to a corresponding flow or pressure control valve to increase the flow of fluid moving through one or more pathways downstream from the valve. If one value of the fluid flow rate moving average exceeds the upper stop threshold value, then a stop command may be generated by the data processing unit to the motor driving the drum to stop rotation of the drum and/or a flow control signal may be generated to a corresponding valve to close the valve. If one value of the fluid flow rate moving average is lower than the lower stop threshold value, then a stop command may be generated by the data processing unit to the motor driving the drum to stop rotation of the drum and/or a flow control signal may be generated to a corresponding valve to close the valve. In any event, a fluid flow rate of a fluid transmitted to one or more nozzles downstream from a corresponding fluid flow sensor may be considered to fall outside of an expected fluid flow rate range if one value (or any predefined number of values) exceeds one or more of the upper warning and stop threshold values or falls below one or more of the lower warning and stop threshold values. Hence, by monitoring the fluid flow data from the fluid flow sensors 18, the data processing unit 38, 38A is able to determine if there are clogs and/or leaks within individual fluid nozzles 12 and/or associated fluid pathways and/or within an individual group of two or more fluid nozzles 12 and/or associated fluid pathways within the drum 4.

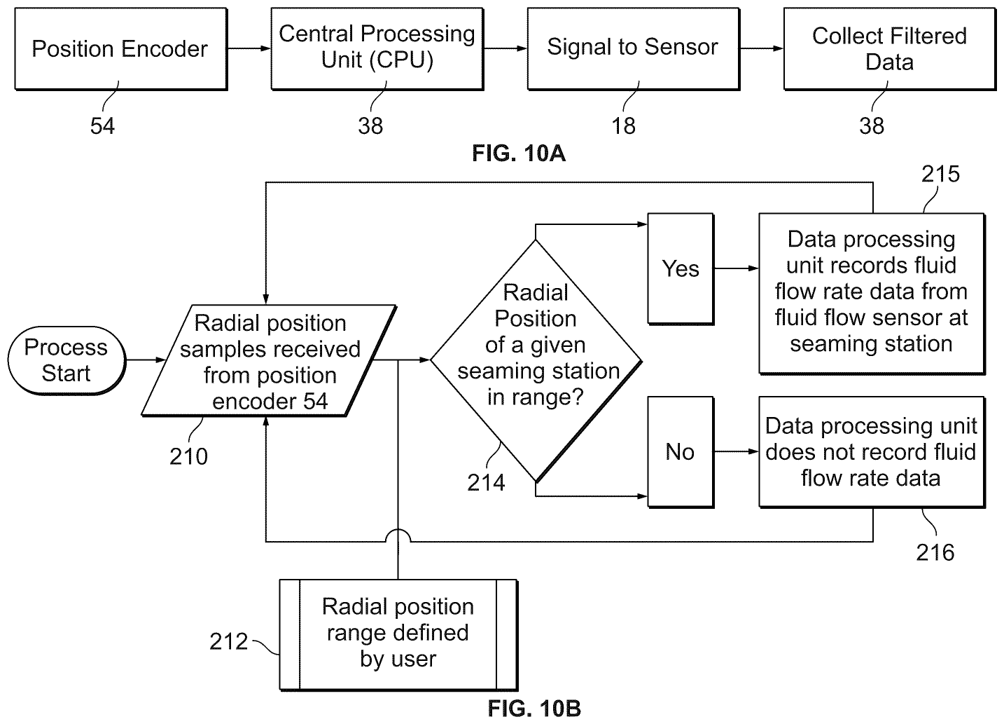

FIGS. 10A and 10B illustrate examples of a filtering process flow chart utilized with the radial pneumatic distribution system 2 to initiate collection and/or processing of data generated by one or more of the fluid flow sensors 18 when a specified angular position of one or more fluid nozzles 12 associated with the one or more fluid flow sensors 18 falls within a user predefined angular data collection range. As noted above, an angular position sensor, for example, a position encoder 54, may be associated with and/or present on the drum 4 and/or dead shaft 24. The position encoder 54 may generate encoder signals, i.e., angular position data, relative to a flag or known predefined point on the drum 4 passing the encoder 54, which encoder 54 may be located at a fixed reference location on the dead shaft 24, such that the encoder signals correspond to an angular position of the drum flag relative to the fixed reference location on the dead shaft 24. The data processing unit(s) 38, 38A are provided with known positions of the one or more seaming stations 32 and, hence, the one or more corresponding fluid nozzles 12 at each seaming station 32, relative to the flag on the drum, which positions of the seaming stations remain constant or fixed relative to the drum flag and, accordingly, the data processing unit(s) 38, 38A are able to determine the angular location of each seaming station and the corresponding one or more fluid nozzles at each seaming station relative to the fixed reference location on the dead shaft 24 as the drum rotates.

As shown in FIGS. 10A and 10B, when the data processing unit(s) 38, 38A receives the angular position data from the position encoder 54, either directly from the position encoder 54 or via the data transfer unit 36, the data processing unit(s) 38, 38A determines the angular location of each seaming station 32 and, hence, its corresponding one or more fluid nozzles 12, relative to the fixed reference location on the dead shaft 24, see steps 210 and 214 in FIG. 10B. When a given seaming station 32 and its corresponding one or more fluid nozzles 12 enter into a user predefined angular data collection range relative to the fixed reference location on the dead shaft 24, see step 212, the data processing unit(s) 38, 38A will trigger the fluid flow sensor(s) associated with the given seaming station 32 to sense the fluid flow rate of the fluid going to the one or more fluid nozzles 12 associated with the seaming station that just entered the user predefined data collection range, i.e., to sense a value, and the fluid flow sensor(s) will then forward the corresponding fluid flow rate data to the data processing unit(s) 38, 38A. If all of the fluid flow sensors 18 continuously forward the fluid flow rate data sensed by all of the fluid flow sensors 18 to the data processing unit(s) 38, 38A, then the data processing unit(s) 38, 38A will start monitoring, storing and/or processing the fluid flow rate data from the seaming station 32 that just entered the user predefined angular data collection range, rather than ignore the fluid flow rate data, see steps 214 and 215 in FIG. 10B. Hence, as each seaming station 32 enters into the user predefined angular data collection range, the data processing unit(s) 38, 38A will begin triggering, monitoring, storing and/or processing the fluid flow rate data from that seaming station's one or more fluid flow sensors 18 and continue to trigger, monitor, store and/or process the fluid flow rate data from that seaming station's one or more fluid flow sensors 18 until the seaming station 32 has moved out of the user predefined angular data collection range, at which point the data processing unit(s) 38, 38A will stop storing and/or processing the fluid flow rate data from that seaming station 32.

The user predefined angular data collection range comprises an angular range relative to the fixed location on the dead shaft 24 such that the data processing unit(s) 38, 38A will only monitor, store and/or process fluid flow rate data from the seaming stations 32 that are located within the user predefined angular data collection range. For example, the user may define the range as corresponding to the angular range of the manifold 52, such that the data processing unit(s) 38, 38A will only monitor, process and/or store fluid flow rate data from seaming stations 32 when those seaming stations 32 are located within the angular data collection range corresponding to the manifold 52. Another angular range may be used for the predefined angular data collection range. Hence, as soon as a seaming station 32 exits the user predefined angular data collection range, the data processing unit(s) 38, 38A will ignore and not process fluid flow rate data from the one or more fluid flow sensors 18 of that seaming station 32, see step 216 in FIG. 10B. Consequently, the number of fluid flow sensor data points that the data processing unit(s) 38, 38A has to process is reduced or filtered as it only monitors and/or processes data from seaming stations 32 located within the user predefined angular data collection range, thereby reducing the amount of processing power required by the data processing unit(s) 38, 38A during monitoring of the data from the fluid flow sensors 18. In FIG. 8B, the peak data of the curves C₁ and C₂ corresponds to a user defined angular data collection range, i.e., the portion or peak data of the curves C₁ and C₂ shown in FIG. 8B was collected when the corresponding seaming station 32 was located within a user defined angular data collection range.

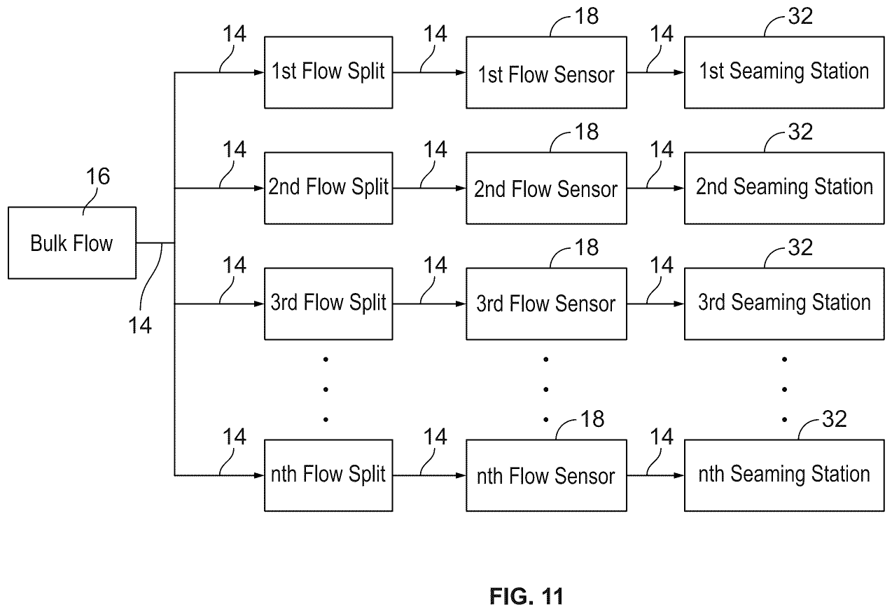

During operation of the drum 4 and the radial pneumatic distribution system 2, an example of a fluid flow pathway from the fluid source 16 into the drum 4 and then distributed to one or more seaming stations 32 is shown in FIG. 11. As shown in FIG. 11, a fluid 14 flows from a fluid source 16, which is located outside of the housing 6 of the drum 4. The fluid flow of fluid 14 then is split or separated into a plurality of fluid pathways by the rotor 28, as shown in FIG. 7A. The fluid flow of the fluid 14 then flows into a plurality of further pathways, each of which may be associated with and monitored by one or more corresponding fluid flow sensors 18, wherein each further fluid flow pathway may be associated with a corresponding seaming station 32 at which point the fluid 14 exits the drum 4 via one or more apertures 40 as shown in FIG. 4.

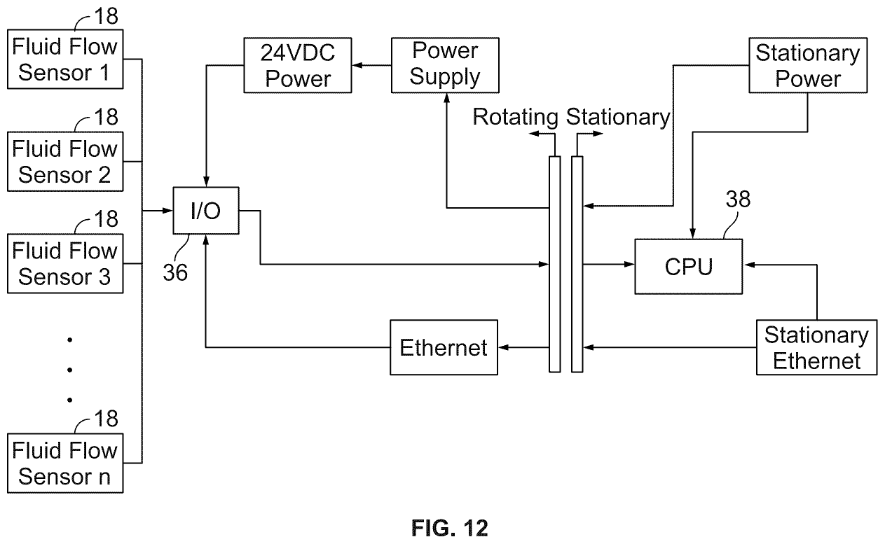

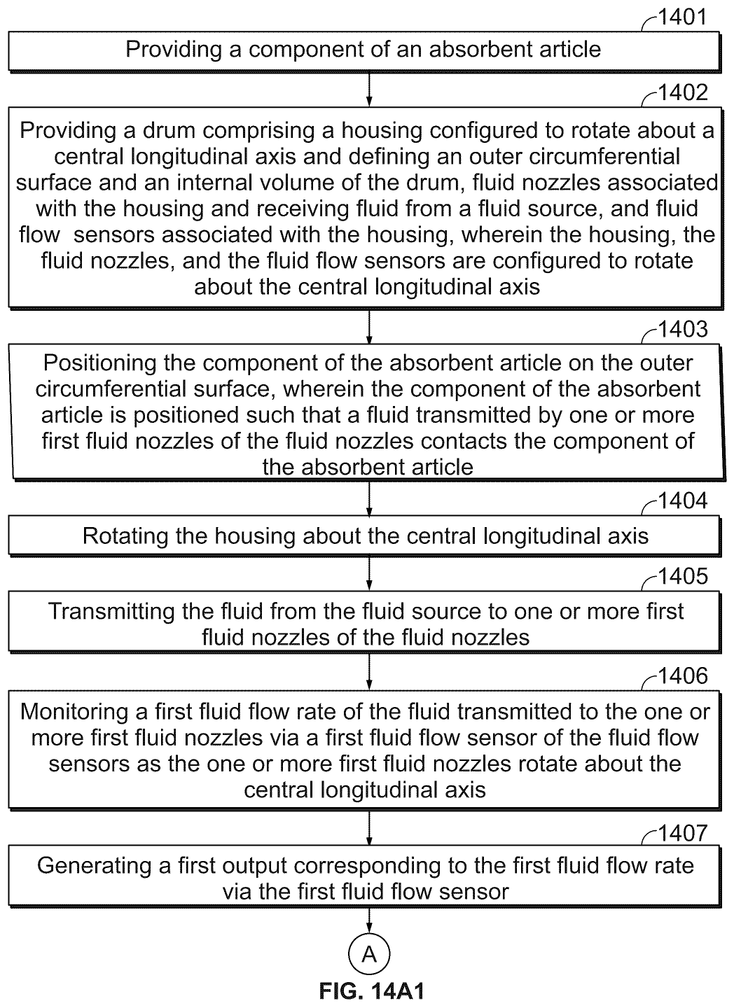

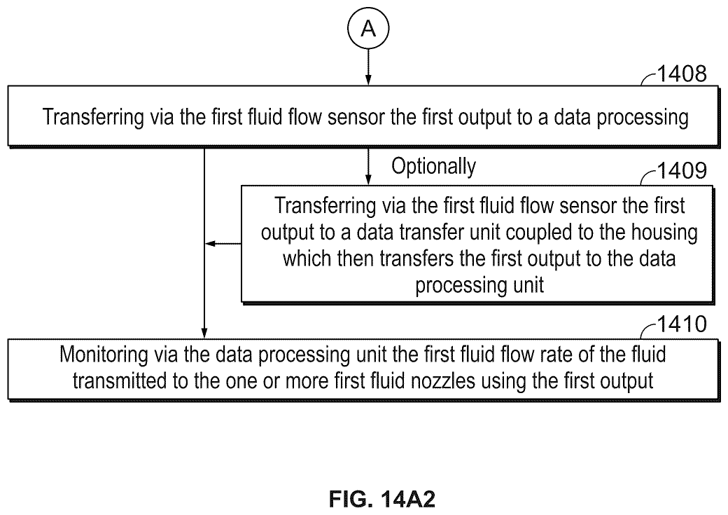

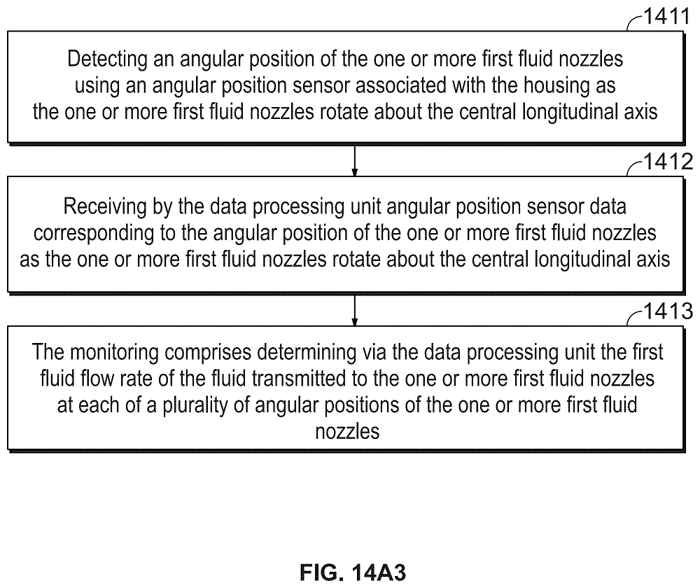

As shown in FIG. 12 and described hereinabove, the radial pneumatic distribution system 2 may comprise one or more stationary, non-rotating, non-moving components, for example, a fluid source 16, a dead shaft 24 with a bore 26, and a data processing unit 38. The radial pneumatic distribution system 2 as shown in FIG. 12, may also include one or more rotating, moving components, for example, a rotor 28, one or more fluid flow sensors 18, one or more fluid nozzles 12, which may be associated with at least one seaming station 32, and one or more data transfer units 36. In one embodiment, as described hereinabove and shown in FIG. 12, a signal from each of one or more sensors, for example, one or more fluid flow sensors 18 may be transferred through one or more data transfer units 36, from the rotating side of the radial pneumatic distribution system 2 to the data processing unit 38 on the stationary side of the radial pneumatic distribution system 2. The signal path of a sensor, for example, a fluid flow sensor 18 from the rotating side to the stationary side of the radial pneumatic distribution system 2 as shown in FIG. 14 may include an I/O device defined by the data transfer unit 36 to a PLC defined by the data processing unit 38, and an ethernet for communications. In one embodiment, as described hereinabove and shown in FIG. 12, a signal from each of one or more sensors, for example, one or more of the fluid flow sensors 18 may be transferred through the one or more data transfer units 36, to the data processing unit 38 on the stationary side of the radial pneumatic distribution system 2. A commercially available rotating coupling can be used to provide energy and/or data acquisition from the rotating side to the static side of the radial pneumatic distribution system 2.

The location of the fluid flow sensor 18 inside of the drum 4 results in fluid flow rate data being monitored, detected, measured, and/or captured for an individual fluid nozzle 12 or an individual group of two or more fluid nozzles 12 in a seaming station 32 within the drum 4 as shown in FIGS. 1 and 2 because the fluid flow sensor 18 is specifically associated with or linked to an individual fluid nozzle 12 or group of fluid nozzles 12 rather than as shown in FIG. 3 where the bulk fluid flow sensor is unable to monitor, detect, measure, and/or capture fluid flow rate data when more than one fluid nozzle 12 is present in the drum 4. As a result, the fluid flow rate data obtained in the drum configuration of the present disclosure is able to identify clogs and/or leaks within individual fluid nozzles 12 and/or associated fluid nozzle pathways and/or within an individual group of two or more fluid nozzles 12 and/or associated fluid nozzle pathways within the drum 4. Further, the configuration of the drum 4 of the present disclosure permits the identification of the specific position of an individual fluid nozzle 12 and/or an individual group of two or more fluid nozzles 12 around the outer circumferential surface 8 of the housing 6 of the drum 4, when an angular position sensor is present, and thus, permits determination as to which fluid nozzle 12 or group of fluid nozzles 12 is malfunctioning or mis-performing, such as is clogged and/or is leaking fluid leading to potential defects in the bonds, for example, side seams, formed and ultimately defects in the absorbent articles produced therefrom.

The radial pneumatic distribution system 2 of the present disclosure provides novel methods for sampling and correcting fluid flow issues within the drum 4. As described herein, in one embodiment, the fluid flow rate data for the all of fluid nozzles 12 within the radial pneumatic distribution system may be monitored, detected, measured, captured, and/or collected and filtered based on the angular position of the fluid nozzles 12 relative to the fixed reference location on the dead shaft 24 as the fluid nozzles 12 rotate about the dead shaft 24. Further, the monitoring, detecting, measuring, capturing, and/or collecting of fluid flow rate data may occur only from fluid nozzles 12 that are present within a user predefined angular data collection range. Further, this process may occur during multiple revolutions of the housing 6 and the fluid nozzles 12 about the central longitudinal axis A1. In addition to the above, in one embodiment, the collection of fluid flow rate data occurs at high speeds, for example, over 10 and/or over 100 and/or over 1000 data points per second, so that a user and/or machine can process the information and determine if any action is needed to correct a malfunctioning fluid nozzle 12.

### Methods of the Present Disclosure

The radial pneumatic distribution system 2 of the present disclosure is useful in the methods described herein. In one example, the radial pneumatic distribution system 2 is useful in a method for determining a fluid flow rate in an apparatus, for example, a drum 4 of the present disclosure.

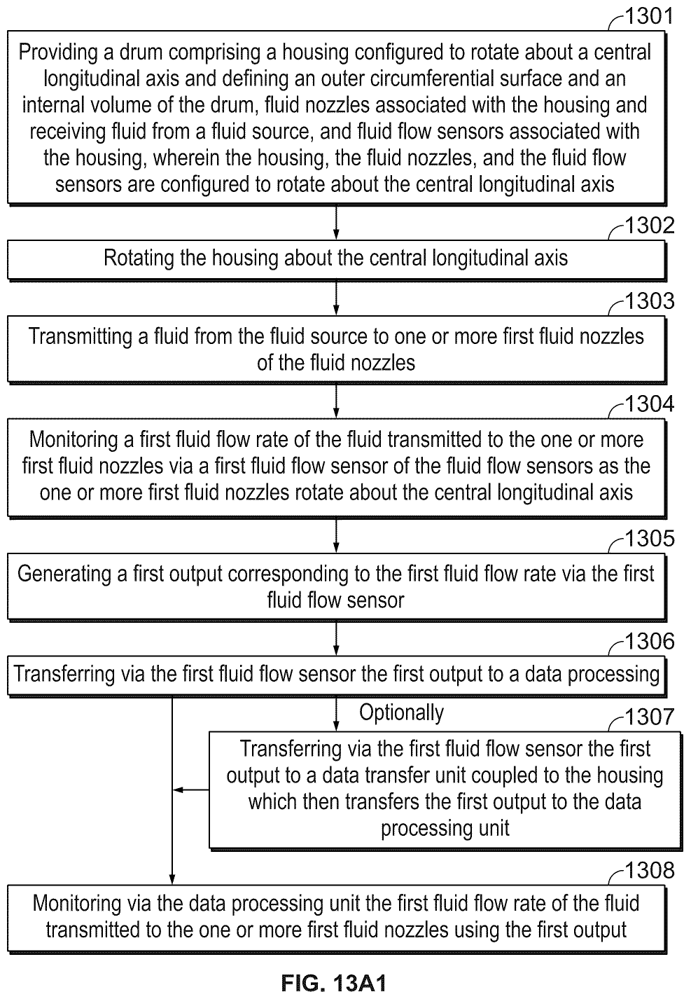

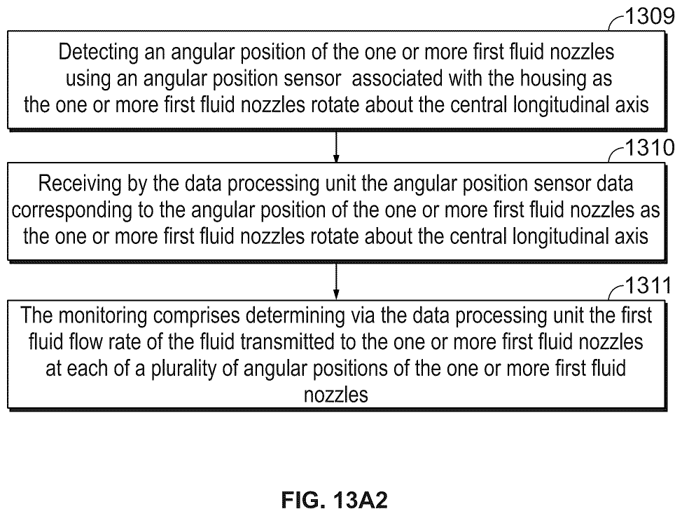

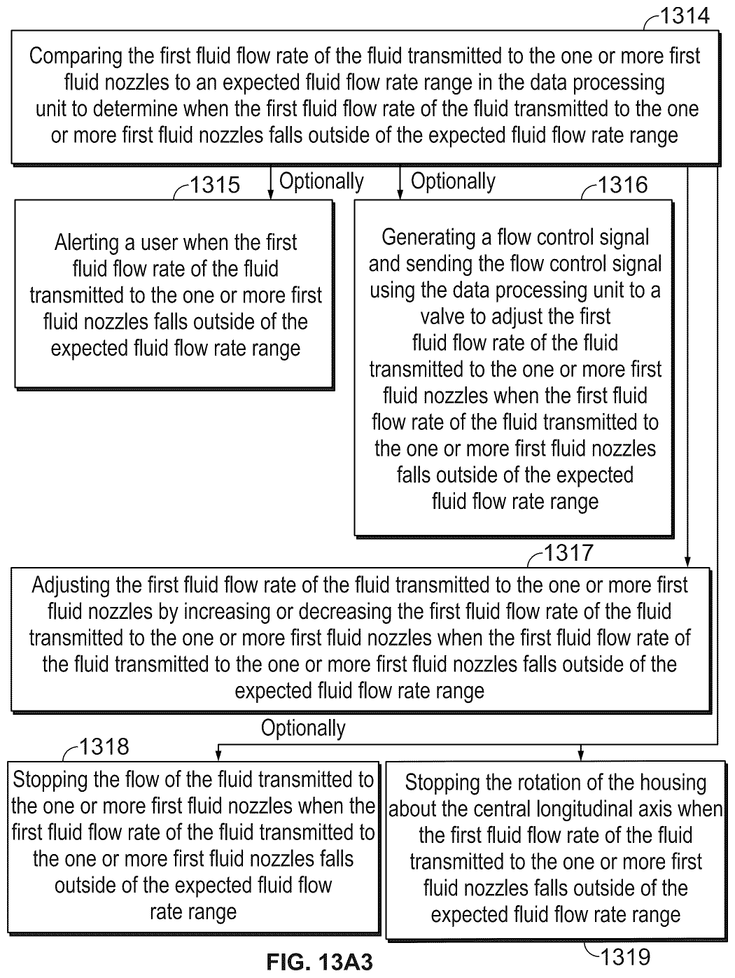

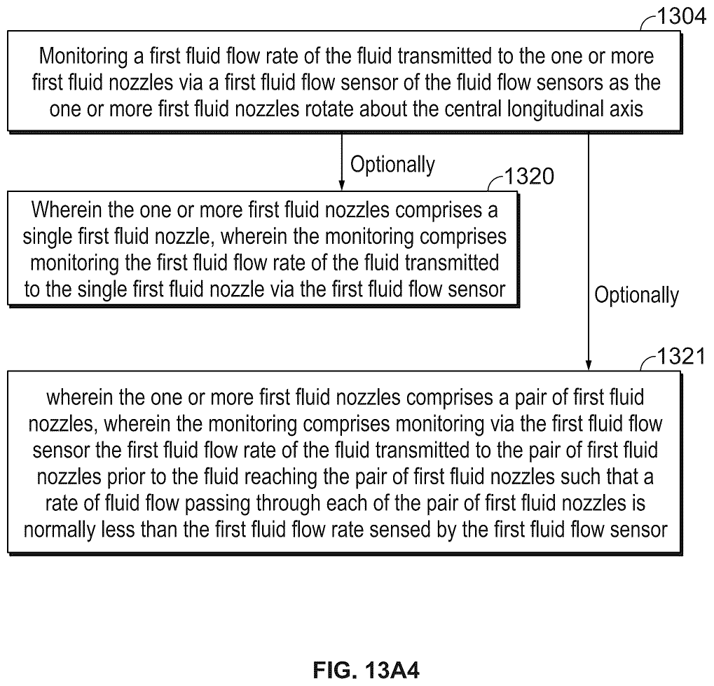

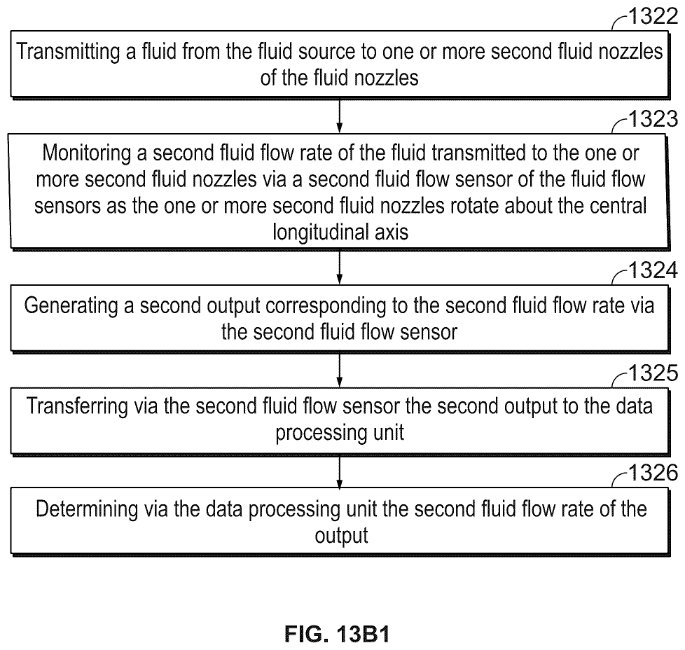

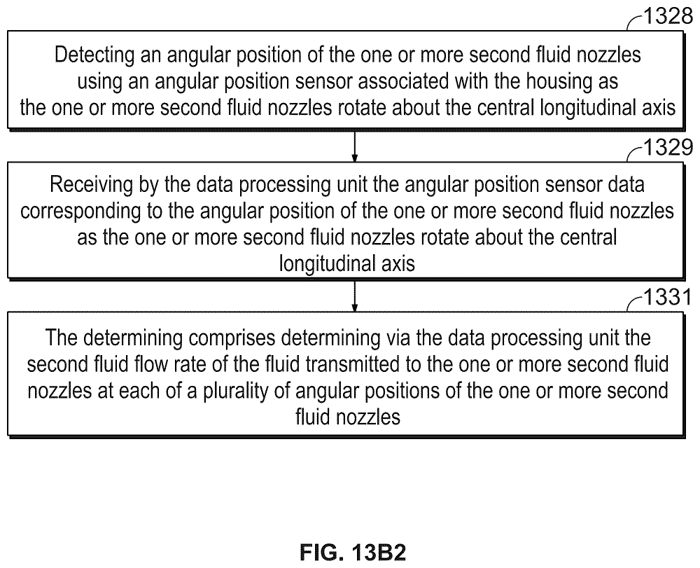

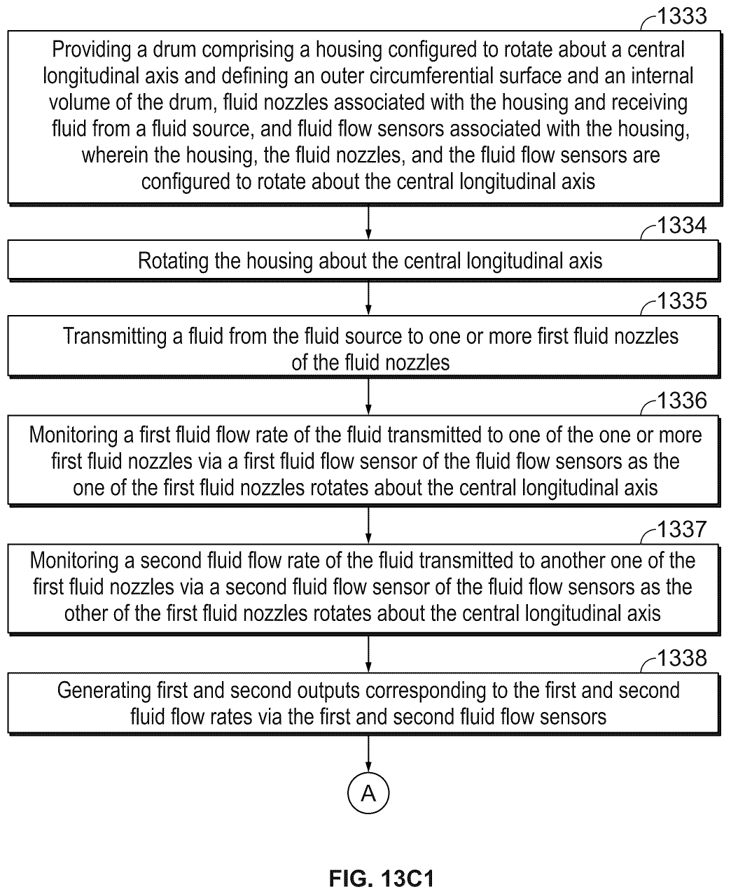

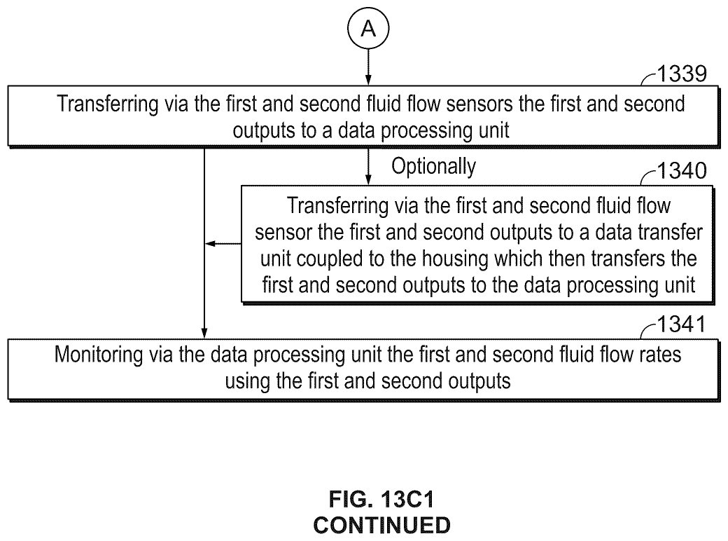

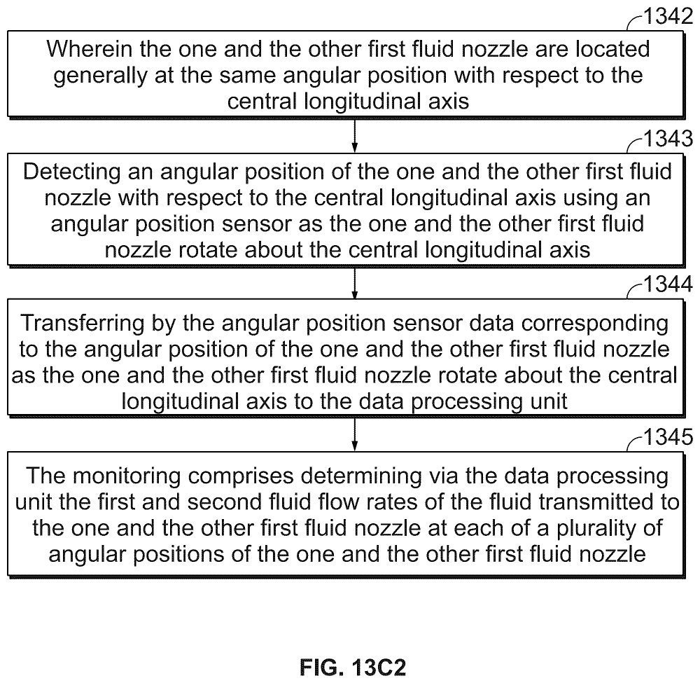

In one embodiment, an example of such a method is shown in FIGS. 13A1-13C2. In FIG. 13A1, the step 1301 includes providing a drum 4 of the present disclosure as described herein. Step 1302 includes rotating the housing 6 of the drum 4 about the drum's central longitudinal axis A1, which may include powering the motor associated with the drum 4. Step 1303 includes transmitting a fluid 14 from the fluid source 16 to and/or through one or more first fluid nozzles 12 of the plurality of fluid nozzles 12. In step 1304, a first fluid flow sensor 18 of the plurality of fluid flow sensors 18 monitors, detects, measures, and/or captures a first fluid flow rate of the fluid 14 transmitted to and/or through the one or more first fluid nozzles 12 as the one or more first fluid nozzles 12 rotate about the central longitudinal axis A1. In step 1305, the first fluid flow sensor 18 generates a first output corresponding to the first fluid flow rate. In step 1306, a data processing unit 38 receives the first output corresponding to the first fluid flow rate from the first fluid flow sensor 18. In optional step 1507, the first output from the first fluid flow sensor 18 may be transferred to a data transfer unit 36 as described herein, which then transfers the first output to the data processing unit 38. The transmission of the first output to the data processing unit 38 may be wireless or wired. In step 1508, the data processing unit 38 monitors a first fluid flow rate of the fluid 14 transmitted to and/or through the one or more first fluid nozzles 12 using the first output.

One or more optional steps in the method may occur, which are illustrated in more detail in FIGS. 13A2-13C2. For example, one optional step as shown in FIG. 13A2 may comprise step 1309, which includes detecting an angular position of the one or more first fluid nozzles using an angular position sensor associated with the housing as the one or more first fluid nozzles rotate about the central longitudinal axis A1. In step 1310, the data processing unit 38 receives the angular position sensor data corresponding to the angular position of the one or more first fluid nozzles as the one or more first fluid nozzles rotate about the central longitudinal axis A1. In step 1311, the monitoring may comprise determining via the data processing unit 38 the first fluid flow rate of the fluid 14 transmitted to the one or more first fluid nozzles 12 at each of a plurality of angular positions of the one or more first fluid nozzles 12.

As shown in FIG. 13A3, the method may further comprise step 1314, where the data processing unit 38 compares the first fluid flow rate of the fluid transmitted to the one or more first fluid nozzles to an expected fluid flow rate range in the data processing unit to determine when the first fluid flow rate of the fluid transmitted to the one or more fluid nozzles falls outside of the expected fluid flow rate range. If the first fluid flow rate falls outside of the fluid flow rate range, then the method may further comprise one or more optional steps. In one embodiment, step 1315 includes alerting a user. In one embodiment, step 1316 includes generating a flow control signal and sending the flow control signal using the data processing unit 38 to a valve to adjust the fluid flow rate of the fluid 14 transmitted to the one or more fluid nozzles 12. In one embodiment, step 1317 includes adjusting the first fluid flow rate of the fluid 14 transmitted to and/or through the one or more first fluid nozzles 12 by increasing or decreasing the first fluid flow rate of the fluid 14 transmitted to and/or through the one or more first fluid nozzles 12. In one embodiment, step 1318 includes stopping the fluid flow of the fluid 14 transmitted to and/or through the one or more first fluid nozzles 12. In one embodiment, step 1319 includes stopping the rotation of the housing 6 of the drum 4 about the central longitudinal axis A1.

As shown in FIG. 13A4, in one embodiment, step 1304 may further comprise step 1320, wherein the one or more first fluid nozzles 12 comprises a single first fluid nozzle 12, wherein monitoring comprises monitoring the first fluid flow rate of the fluid 14 transmitted to the single first fluid nozzle 12 via the first fluid flow sensor 18. In one embodiment, step 1304 may further comprise step 1321, wherein the one or more first fluid nozzles 12 comprises a pair of first fluid nozzles 12, wherein the monitoring comprises monitoring via the first fluid flow sensor 18 the first fluid flow rate of the fluid 14 transmitted to the pair of first fluid nozzles 12 prior to the fluid 14 reaching the pair of first fluid nozzles 12 such that a rate of fluid flow passing through each of the pair of first fluid nozzles 12 is normally less than the first fluid flow rate sensed, for example, detected and/or measured, by the first fluid flow sensor 18.

In one embodiment, the optional steps in the method may further include the optional steps shown FIG. 13B1. As shown in FIG. 13B1, the method may further comprise step 1322, which includes transmitting the fluid 14 from the fluid source 16 to and/or through one or more second fluid nozzles 12 of the fluid nozzles 12. Step 1323 may use a second fluid flow sensor 18 of the fluid flow sensors 18 to monitor, detect, measure, and/or capture a second fluid flow rate of the fluid 14 transmitted to and/or through the one or more second fluid nozzles 12 as the one or more second fluid nozzles 12 rotate about the central longitudinal axis A1. In step 1324, the second fluid flow sensor 18 generates a second output corresponding to the second fluid flow rate. The second fluid flow sensor 18 may then transfer, directly or indirectly, the second output to the data processing unit 38 as shown in step 1325. In step 1326, the data processing unit 38 determines a second fluid flow rate of the second fluid flow of the fluid 14 transmitted to and/or through the one or more second fluid nozzles 12 using the second output.

As shown in FIG. 13B2, the method may further comprise detecting an angular position of the one or more second fluid nozzles associated with the housing using an angular position sensor associated with housing as the one or more second fluid nozzles rotate about the central longitudinal axis A1 as shown in step 1328. In step 1329, the data processing unit 38 may receive the angular position sensor data corresponding to the angular position of the one or more second fluid nozzles 12 as the one or more second fluid nozzles rotate about the central longitudinal axis A1. In step 1331, the data processing unit 38 may determine the second fluid flow rate of the fluid 14 transmitted to and/or through the one or more second fluid nozzles 12 at each of a plurality of angular positions of the one or more second fluid nozzles 12.

As shown in FIG. 13C1, the step 1333 includes providing a drum 4 of the present disclosure as described herein. Step 1334 includes rotating the housing 6 of the drum 4 about the central longitudinal axis A1, which may include powering the motor associated with the drum 4. Step 1335 includes transmitting a fluid 14 from the fluid source 16 to and/or through one or more first fluid nozzles 12 of the fluid nozzles 12. In step 1336, a first fluid flow sensor 18 of the fluid flow sensors 18 monitors, detects, measures, and/or captures a first fluid flow rate of the fluid 14 transmitted to and/or through one of the one or more first fluid nozzles 12 as the one of the first fluid nozzles 12 rotates about the central longitudinal axis A1. In step 1337, a second fluid flow sensor 18 of the fluid flow sensors 18 monitors, detects, measures, and/or captures a second fluid flow rate of the fluid 14 transmitted to and/or through another of the one or more first fluid nozzles 12 as the other of the first fluid nozzles 12 rotates about the central longitudinal axis A1. In step 1338, the first and second fluid flow sensors 18 generate first and second outputs corresponding to the first and second fluid flow rates. In step 1339, the first and second fluid flow sensors 18 transfer the first and second outputs to a data processing unit 38 as described herein. In optional step 1340, the first and second fluid flow sensors 18 may transfer the first and second outputs to a data transfer unit 36 as described herein, which then transfers the first and second outputs to the data processing unit 38. The transmission of the first and second outputs to the data processing unit 38 may be wireless or wired. In step 1341, the data processing unit 38 monitors the first and second fluid flow rates using the first and second outputs.

As shown in FIG. 13C2, the method may further comprise additional optional steps, for example, steps 1342-1347 when the one and the other first fluid nozzle 12 are located generally at the same angular position with respect to the central longitudinal axis A1. Step 1343 includes detecting an angular position of the one and the other first fluid nozzle using an angular position sensor, which may be associated with the drum and/or a motor that drives the drum, as the one and the other first fluid nozzle rotate about the central longitudinal axis A1. In step 1344, the angular position sensor transfers data corresponding to the angular position of the one and the other first fluid nozzle to the data processing unit 38. In step 1345, the data processing unit 38 determines the first and second fluid flow rates of the fluid 14 transmitted to and/or through the one and the other first fluid nozzle 12 at each of a plurality of angular positions of the one and the other first fluid nozzle 12.

In one embodiment, an example of a method, which may be used for measuring a fluid flow rate in a drum used for manufacturing absorbent articles, such as diaper pants, is shown in FIGS. 14A1-14A3. In FIG. 14A1, the step 1401 includes providing a component of an absorbent article. Step 1402 includes providing a drum 4 of the present disclosure as described herein. Step 1403 includes positioning the component of the absorbent article on the outer circumferential surface 8 of the housing 6, wherein the component of the absorbent article is positioned such that a fluid 14 transmitted by one or more first fluid nozzles 12 of the fluid nozzles 12 contact the component of the absorbent article. Step 1404 includes rotating the housing 6 of the drum 4 about the central longitudinal axis A1, which may include powering the motor associated with the drum 4. A fluid 14 is transmitted from the fluid source 16 to and/or through one or more first fluid nozzles 12 of the fluid nozzles 12 in step 1405. In step 1406, a first fluid flow sensor 18 of the fluid flow sensors 18 monitors, detects, measures, and/or captures a first fluid flow rate of the fluid 14 transmitted to and/or through the one or more first fluid nozzles 12 as the one or more first fluid nozzles 12 rotate about the central longitudinal axis A1. In step 1407, the first fluid flow sensor 18 generates a first output corresponding to the first fluid flow rate. In step 1408, the first fluid flow sensor 18 transfers the first output to a data processing unit 38 as described herein. In optional step 1409, the first fluid flow sensor 18 may transfer the first output to a data transfer unit 36 as described herein, which then transfers the first output to the data processing unit 38. The transmission of the first output to the data processing unit 38 may be wireless or wired. In step 1410, the data processing unit 38 monitors a first fluid flow rate of the fluid 14 transmitted to and/or through the one or more first fluid nozzles 12 using the first output.

One or more optional steps in the method may occur, which are illustrated in more detail in FIG. 14A3. For example, one optional step as shown in FIG. 14A3 may comprise step 1411, which includes detecting an angular position of the one or more first fluid nozzles using an angular position sensor associated with the housing as the one or more first fluid nozzles rotate about the central longitudinal axis A1. In step 1412, the data processing unit receives the angular position sensor data corresponding to the angular position of the one or more first fluid nozzles as the one or more first fluid nozzles rotate about the central longitudinal axis A1. In step 1413, the monitoring may comprise determining via the data processing unit 38 the first fluid flow rate of the fluid 14 transmitted to the one or more first fluid nozzles 12 at each of a plurality of angular positions of the one or more first fluid nozzles 12

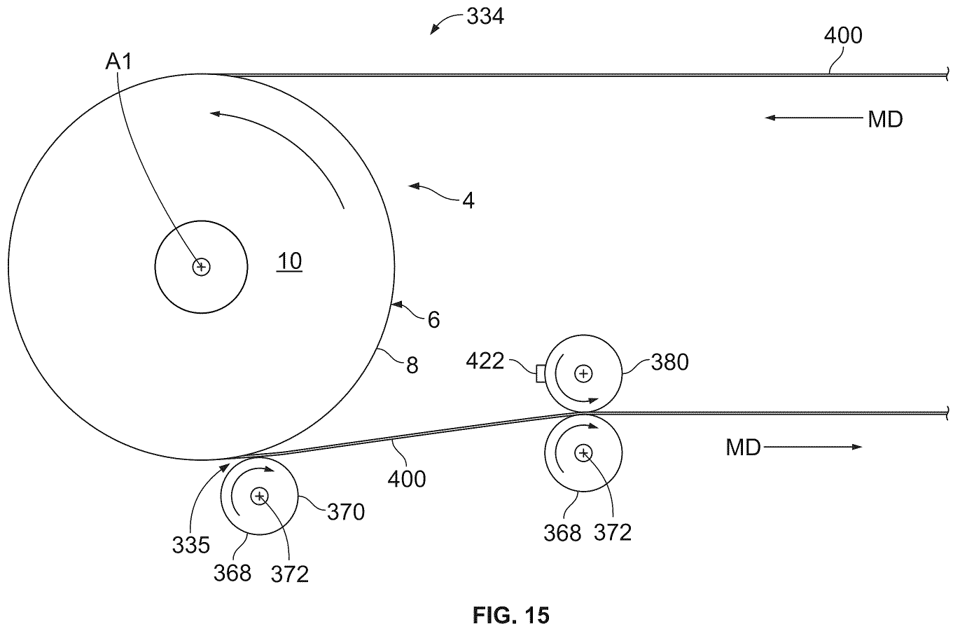

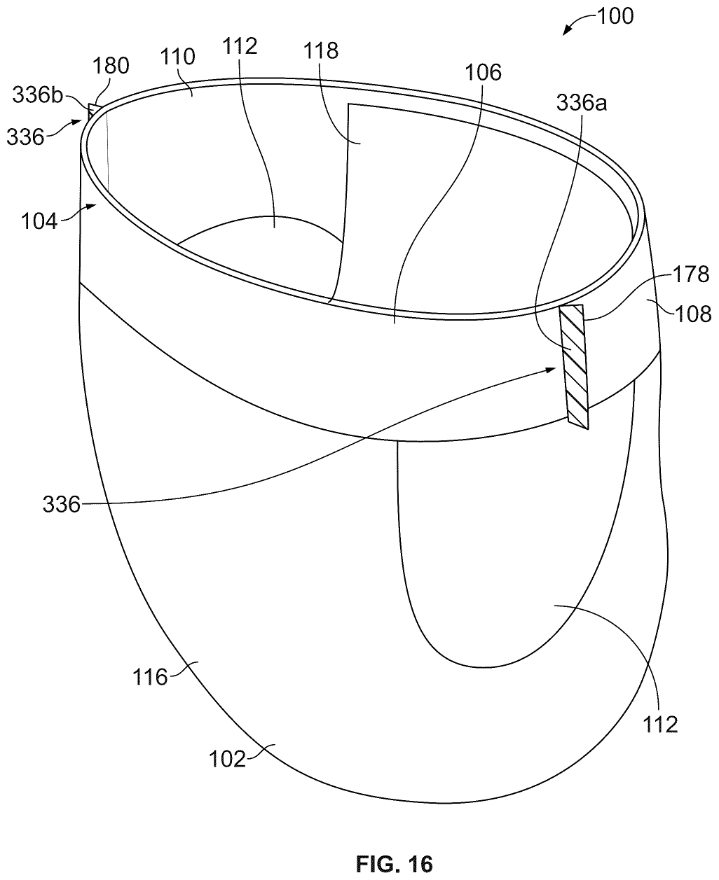

In one embodiment, the radial pneumatic system 2 of the present disclosure may be utilized for creating bonds in webs 20, 22, for example, components of absorbent articles in an absorbent article making machine. With reference to FIGS. 15-16, the absorbent article making machine may include a bonder module 334 to create bonds 336, such as seams, for example, side seams 178, 180. The bonder module 334 may be configured to intermittently bond, for example, seam the webs 20, 22, by directing a jet of fluid 14, for example, heated air to at least partially melt an overlap area of the webs 20, 22, and compressing the melted overlap area between an outer circumferential surface 370 of an anvil roll 368 and optionally, a pressing member 380.

FIG. 16 shows an example of an absorbent article, for example, a belt-type pant 100 that can be manufactured utilizing the radial pneumatic distribution system 2 of the present disclosure described herein. The belt-type pant 100 comprises a chassis 102 and a ring-like elastic belt 104 that comprises a first side seam 178 formed from bond 336a and a second side seam 180 formed from bond 336b.

The belt-type pant 100 comprising the ring-like elastic belt 104 is provided to consumers in a configuration wherein the front waist region 116 and the back waist region 118 are connected to each other as packaged, prior to being applied to the wearer. As such, the belt-type pant 100 may have a continuous perimeter waist opening 110 and continuous perimeter leg openings 112 such as shown in FIG. 16. The ring-like elastic belt 104 is defined by a front belt 106 connected with a back belt 108 at side seams 178, 180.

Referring to FIG. 16, in one embodiment, the first and second side seams 178, 180, which join the lateral edges of the front belt 106 and the back belt 108 are made of material of an elastic laminate for forming the front belt 106 and the back belt 108. In one embodiment, the side seams 178, 180, are made by a hot air bond 336a, 336b, respectively, which at least partially melts the material of the elastic laminate forming the front belt 106 and the back belt 108. The side seams 178, 180, may have continuity of the melted material along the substantial entirety of its longitudinal dimension. As discussed in further detail below, the side seams 178, 180, herein are formed by hot air bonding. Hot air bonding is advantageous in that the process may be conducted in high speed to form a reliable strong seam. It is desired that the side seam is strong enough to tolerate normal usage conditions, namely does not fail when stretched upon application, or after the article is loaded. On the other hand, it is also desired that the seam is easy to open after use, namely possible to tear by hand along the longitudinal dimension for removal from the wearer.

As shown in FIG. 15, the bonder module 334 includes the drum 4 and methods of the present disclosure as shown and described herein.

The bonder module 334 includes the drum 4 and a compressing stage 335 located adjacent to the drum 4. The drum 4 may comprise one or more seaming stations (not shown in FIG. 15) at which one or more portions of the continuous advancing component 400 comprising webs 20, 22, are subjected to the fluid 14 to at least partially melt the webs 20, 22, at the one or more seaming stations. The continuous advancing component 400 may be rotated about the drum 4 in order to provide sufficient time for the melting, such that the melted portion may be compressed at the compressing stage 335 to provide the side seam 178, 180 of the resulting absorbent article to have continuity of melted material along the substantial entirety of its longitudinal dimension. The fluid nozzles 12 within the drum 4 may be actively directing fluid 14 only when communicating with the manifold 52 and may not be actively directing fluid 14 when not communicating with the manifold 52, as described above. The manifold 52 is located and configured such that when one or more seaming stations 32 are positioned next to or directly adjacent to the moving webs 20, 22, the nozzles in those seaming stations 32 communicate with the manifold 52 to receive fluid to actively direct fluid to the webs 20, 22. The nozzles in the seaming stations may no longer communicate with the manifold when those seaming stations rotate away from the webs 20, 22.

As shown in FIG. 15, the compressing stage 335 may be located on the drum 4 and/or optionally, shortly after leaving the drum 4. The compressing stage 335 may comprise an anvil roll 368 having an outer circumferential surface 370 and configured to engage with the outer circumferential surface 8 of the housing 6 of the drum 2 prior to the continuous advancing component 400 leaving the outer circumferential surface 8 of the housing 6 wherein the engagement is configured to occur immediately before the continuous advancing component 400 leaves the drum 4. In addition, or alternatively, the bonder module 334 may further comprise a compressing stage 335 comprising an anvil roll 368 and a pressing member 380 that is adjacent to, but spaced away from the drum 4. The anvil roll 368 includes an outer circumferential surface 370 and is adapted to rotate about an axis of rotation 372.

The pressing member 380, when present, may comprise a pair of projections 422 for engaging with the outer circumferential surface 370 of the anvil roll 368 for forming a pair of side seams 178, 180, adjacent to each other. By providing the compressing stage 335 independent of the drum 4, the anvil roll 368 and the pressing member 380 may each be adjusted according to the type of component to bond and/or seam. This is advantageous in that components made of various types of webs and various sizes may be seamed without the need to fabricate a new drum 4. The outer circumferential surface 370 of the anvil roll 368 and the projections 422 of the pressing member 380 to engage with the outer circumferential surface 370 may have configurations for providing varying bonding pressure.

### Combinations

A. A drum comprising a housing that defines an outer circumferential surface and an internal volume of the drum, wherein a plurality of fluid nozzles and a plurality of fluid flow sensors each in fluid communication with at least one of the plurality of fluid nozzles are associated with the housing and wherein the outer circumferential surface comprises a plurality of apertures associated with the plurality of fluid nozzles.
B. The drum according to Paragraph A, wherein the plurality of fluid nozzles comprises two or more groups of fluid nozzles supported by the housing, wherein the two or more groups of fluid nozzles are spaced apart from one another about the housing and wherein each of the two or more groups of fluid nozzles is associated with one of the plurality of apertures.
C. The drum according to Paragraph B, wherein the plurality of fluid flow sensors comprises two or more groups of fluid flow sensors, each of the two or more groups of fluid flow sensors being associated with a corresponding group of fluid nozzles.
D. The drum according to Paragraph A, wherein the plurality of fluid nozzles comprises a first group of fluid nozzles comprising a first fluid nozzle and a second fluid nozzle, wherein one of the plurality of fluid flow sensors is associated with the first fluid nozzle and the second fluid nozzle.
E. The drum according to Paragraph A, wherein the plurality of fluid nozzles comprises a first group of fluid nozzles comprising a first fluid nozzle and a second fluid nozzle, wherein the plurality of fluid flow sensors comprises a first fluid flow sensor associated with the first fluid nozzle and configured to detect a fluid flow rate of a fluid flowing to the first fluid nozzle and a second fluid flow sensor associated with the second fluid nozzle and configured to detect a fluid flow rate of a fluid flowing to the second fluid nozzle.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

What is claimed is:

1. A method comprising:
providing a drum comprising a housing configured to rotate about a central longitudinal axis and defining an outer circumferential surface and an internal volume of the drum, fluid nozzles associated with the housing and receiving fluid from a fluid source, and fluid flow sensors associated with the housing, wherein the housing, the fluid nozzles, and the fluid flow sensors are configured to rotate about the central longitudinal axis;
rotating the housing about the central longitudinal axis;
transmitting a fluid from the fluid source to one or more first fluid nozzles of the fluid nozzles;
monitoring a first fluid flow rate of the fluid transmitted to the one or more first fluid nozzles via a first fluid flow sensor of the fluid flow sensors as the one or more first fluid nozzles rotate about the central longitudinal axis;
generating a first output corresponding to the first fluid flow rate via the first fluid flow sensor;
receiving by a data processing unit the first output corresponding to the first fluid flow rate from the first fluid flow sensor; and
monitoring via the data processing unit the first fluid flow rate of the fluid transmitted to the one or more first fluid nozzles using the first output.

2. The method according to claim 1, further comprising transferring the first output from the first fluid flow sensor to a data transfer unit coupled to the housing prior to transferring the first output to the data processing unit.

3. The method according to claim 1, wherein the method further comprises:
detecting an angular position of the one or more first fluid nozzles using an angular position sensor associated with the housing as the one or more first fluid nozzles rotate about the central longitudinal axis;
receiving by the data processing unit the angular position sensor data corresponding to the angular position of the one or more first fluid nozzles as the one or more first fluid nozzles rotate about the central longitudinal axis; and
the monitoring comprises determining via the data processing unit the first fluid flow rate of the fluid transmitted to the one or more first fluid nozzles at each of a plurality of angular positions of the one or more first fluid nozzles that fall within a predefined angular data collection range.

4. The method according to claim 3, wherein the method further comprises:
comparing the first fluid flow rate of the fluid transmitted to the one or more first fluid nozzles to an expected fluid flow rate range in the data processing unit to determine when the first fluid flow rate of the fluid transmitted to the one or more first fluid nozzles falls outside of the expected fluid flow rate range.

5. The method according to claim 4, wherein the method further comprises:
alerting a user when the first fluid flow rate of the fluid transmitted to the one or more first fluid nozzles falls outside of the expected fluid flow rate range.

6. The method according to claim 4, wherein the method further comprises:
generating a flow control signal and sending the flow control signal using the data processing unit to a valve to adjust the first fluid flow rate of the fluid transmitted to the one or more first fluid nozzles when the first fluid flow rate of the fluid transmitted to the one or more first fluid nozzles falls outside of the expected fluid flow rate range.

7. The method according to claim 4, wherein the method further comprises:
adjusting the first fluid flow rate of the fluid transmitted to the one or more first fluid nozzles by increasing or decreasing the first fluid flow rate of the fluid transmitted to the one or more first fluid nozzles when the first fluid flow rate of the fluid transmitted to the one or more first fluid nozzles falls outside of the expected fluid flow rate range.

8. The method according to claim 4, wherein the method further comprises:
stopping the flow of the fluid transmitted to the one or more first fluid nozzles when the first fluid flow rate of the fluid transmitted to the one or more first fluid nozzles falls outside of the expected fluid flow rate range.

9. The method according to claim 4, wherein the method further comprises:
stopping the rotation of the housing about the central longitudinal axis when the first fluid flow rate of the fluid transmitted to the one or more first fluid nozzles falls outside of the expected fluid flow rate range.

10. The method according to claim 1, wherein the one or more first fluid nozzles comprises a single first fluid nozzle, wherein the monitoring comprises monitoring the first fluid flow rate of the fluid transmitted to the single first fluid nozzle via the first fluid flow sensor.

11. The method according to claim 1, wherein the one or more first fluid nozzles comprises a pair of first fluid nozzles, wherein the monitoring comprises monitoring via the first fluid flow sensor the first fluid flow rate of the fluid transmitted to the pair of first fluid nozzles prior to the fluid reaching the pair of first fluid nozzles such that a rate of fluid flow passing through each of the pair of first fluid nozzles is normally less than the first fluid flow rate sensed by the first fluid flow sensor.

12. The method according to claim 1, wherein the method further comprises:
transmitting the fluid to one or more second fluid nozzles of the one or more fluid nozzles;
monitoring a second fluid flow rate of the fluid transmitted to the one or more second fluid nozzles via a second fluid flow sensor of the fluid flow sensors as the one or more second fluid nozzles rotate about the central longitudinal axis;
generating a second output corresponding to the second fluid flow rate via the second fluid flow sensor;
transferring the second output to the data processing unit; and
determining via the data processing unit the second fluid flow rate of the fluid transmitted to the one or more second fluid nozzles using the second output.

13. The method according to claim 12, wherein the method further comprises:
detecting an angular position of the one or more second fluid nozzles using the angular position sensor associated with the housing as the one or more second fluid nozzles rotate about the central longitudinal axis;
receiving by the data processing unit the angular position sensor data corresponding to the angular position of the one or more second fluid nozzles as the one or more second fluid nozzles rotate about the central longitudinal axis; and
the determining comprises determining via the data processing unit the second fluid flow rate of the fluid transmitted to the one or more second fluid nozzles at each of a plurality of angular positions of the one or more second fluid nozzles that fall within a predefined angular data collection range.

14. A method comprising:
providing a drum comprising a housing configured to rotate about a central longitudinal axis and defining an outer circumferential surface and an internal volume, fluid nozzles associated with the housing and receiving fluid from a fluid source, and fluid flow sensors associated with the housing, wherein the housing, the fluid nozzles, and the fluid flow sensors are configured to rotate about the central longitudinal axis;
rotating the housing about the central longitudinal axis;
transmitting a fluid from the fluid source to one or more first fluid nozzles of the fluid nozzles;
monitoring a first fluid flow rate of the fluid transmitted to one of the first fluid nozzles via a first fluid flow sensor of the fluid flow sensors as the one of the first fluid nozzles rotates about the central longitudinal axis;
monitoring a second fluid flow rate of the fluid transmitted to another one of the first fluid nozzles via a second fluid flow sensor of the fluid flow sensors as the other of the first fluid nozzles rotates about the central longitudinal axis;
generating first and second outputs corresponding to the first and second fluid flow rates via the first and second fluid flow sensors;
receiving by a data processing unit the first and second outputs; and
monitoring via the data processing unit first and second fluid flow rates using the first and second outputs.

15. The method according to claim 14, wherein the one and the other first fluid nozzle are located generally at the same angular position, wherein the method further comprises:
detecting an angular position of the one and the other first fluid nozzle using an angular position sensor associated with the housing as the one and the other first fluid nozzle rotate about the central longitudinal axis;
receiving by the data processing unit the angular position sensor data corresponding to the angular position of the one and the other first fluid nozzle as the one and the other first fluid nozzle rotate about the central longitudinal axis;
the monitoring comprises determining via the data processing unit the first and second fluid flow rates of the fluid transmitted to the one and the other first fluid nozzle at each of a plurality of angular positions of the one and the other first fluid nozzle that fall within a predefined angular data collection range.

16. A method comprising:
providing a component of an absorbent article;
providing a drum comprising a housing configured to rotate about a central longitudinal axis and defining an outer circumferential surface and an internal volume of the drum, fluid nozzles associated with the housing and receiving fluid from a fluid source, and fluid flow sensors associated with the housing, wherein the housing, the fluid nozzles, and the fluid flow sensors are configured to rotate about the central longitudinal axis;
positioning the component of the absorbent article on the outer circumferential surface, wherein the component of the absorbent article is positioned such that fluid transmitted by one or more first fluid nozzles of the fluid nozzles contacts the component of the absorbent article;
rotating the housing about the central longitudinal axis;
transmitting a fluid from the fluid source to the one or more first fluid nozzles;
monitoring a first fluid flow rate of the fluid transmitted to the one or more first fluid nozzles via a first fluid flow sensor of the fluid flow sensors as the one or more first fluid nozzles rotate about the central longitudinal axis;
generating a first output corresponding to the first fluid flow rate via the first fluid flow sensor;
transferring the first output to a data processing unit; and
monitoring via the data processing unit a first fluid flow rate of the fluid transmitted to the one or more first fluid nozzles using the first output.

17. The method according to claim 16, wherein the method further comprises:
detecting an angular position of the one or more first fluid nozzles using an angular position sensor associated with the housing as the one or more first fluid nozzles rotate about the central longitudinal axis;
transferring via the angular position sensor data corresponding to the angular position of the one or more first fluid nozzles as the one or more first fluid nozzles rotate about the central longitudinal axis to the data processing unit; and
the monitoring comprises determining via the data processing unit the first fluid flow rate of the fluid transmitted to the one or more first fluid nozzles at each of a plurality of angular positions of the one or more first fluid nozzles that fall within a predefined angular data collection range.

18. The method according to claim 16, wherein the component of the absorbent article comprises at least one of a first nonwoven, a second nonwoven, or an elastomeric material.
